(19) United States
(12) Patent Application Publication
(10) Pub. No.: US 2026/0152799 A1
(43) Pub. Date: Jun. 4, 2026

(54) TUMOUR STRATIFICATION FOR RESPONSIVENESS TO AN IMMUNE CHECKPOINT INHIBITOR

(71) Applicants: MEMORIAL SLOAN-KETTERING CANCER CENTER, New York, NY (US); CANCER RESEARCH TECHNOLOGY LIMITED, London (GB)

(72) Inventors: Payam GAMMAGE, London (GB); Eduard REZNIK, New York, NY (US)

(21) Appl. No.: 19/123,257

(22) PCT Filed: Oct. 24, 2024
(86) PCT No.: PCT/GB2023/052786

§ 371 Date: Apr. 22, 2025

Related U.S. Application Data
(60) Provisional application No. 63/380,599, filed on Oct. 24, 2022.

(30) Foreign Application Priority Data
Nov. 8, 2022 (GB) 2216644.1

(51) Int. Cl. C12Q 1/6886

(52) U.S. Cl. CPC C12Q 1/6886; C12Q 2600/106; C12Q 2600/156

(57) ABSTRACT

The present invention relates to methods of predicting whether a subject having a cancer or a pre-cancer would benefit from treatment with an immune checkpoint inhibitor, as well as methods for treating subjects determined to be likely to benefit from such treatment.

The present invention relates to methods of predicting whether a subject having a cancer or a pre-cancer would benefit from treatment with an immune checkpoint inhibitor, as well as methods for treating subjects determined to be likely to benefit from such treatment.

### BACKGROUND

Cancer immunotherapy involves the attack of cancer cells by a patient's immune system. Regulation and activation of T lymphocytes depends on signaling by the T-cell receptor and co-signaling receptors that deliver positive or negative signals for activation. Immune responses by T cells are controlled by a balance of costimulatory and inhibitory signals, called immune checkpoints.

Immunotherapy with immune checkpoint inhibitors is revolutionising cancer therapy. However, some patients show little or no improvement with immune checkpoint inhibitor therapies. Accordingly, methods for better patient stratification to determine which patients are likely to benefit from such treatment are still needed. The present invention aims to address this need at least in part.

### BRIEF SUMMARY OF THE DISCLOSURE

The present invention is based on the inventors' surprising finding that cancer cells with heteroplasmic deleterious mutations in mitochondrial DNA (mtDNA) may have an altered tumour microenvironment. Specifically, the present inventors have shown that cancer cells with a deleterious mtDNA mutation present at a high mutation load are associated with different immune cell populations being present in the tumour microenvironment. As discussed in the Examples section of the present application, the inventors have found that microenvironments of tumours comprising such cancer cells are enriched in Natural Killer (NK) cells, monocytes, CD4+ T cells, and interferon-stimulated gene (ISG) expressing immune cells, but have reduced macrophage levels and tumour-associated neutrophil levels, as compared to the microenvironments of tumours with cancer cells that have no or low deleterious mtDNA mutation load.

The inventors believe that the presence of a deleterious mtDNA mutation load alters the cancer cell metabolism in a way that alters the tumour microenvironment making it conducive to infiltration by certain populations of immune cells. As discussed in the Examples section of the present disclosure, the inventors have identified that cancer cells with heteroplasmic mutations in the MT-ND5 gene showed increased levels of reduced nicotinamide adenine dinucleotide (NADH), leading to disrupted NAD+:NADH ratio and altered cellular redox balance. This may result in reverse flux of Malate Dehydrogenase 2 (MDH2) and accumulation of cytosolically derived malate via Malate Dehydrogenase 1 (MDH1). The increased MDH1 activity may drive glycolysis and result in excess glucose consumption and excess lactate release. Surprisingly, the inventors found that, despite these changes, oxygen consumption and ATP synthesis remained unaffected at a 60% mutation load (also referred to herein as “variant allele frequency” or “VAF”), although the inventors believe that these parameters would be impacted by a higher mutation load. Without wishing to being bound by this hypothesis, the inventors believe that these metabolic changes promote the recruitment and/or survival of specific immune cell types (such as those mentioned above) into the tumour which are less sensitive to altered redox status (for example an altered glucose to lactate ratio). This reduced sensitivity, the inventors hypothesise, could be due to the cells' preferential utilisation of lactate as a carbon fuel source or lower dependency on glucose.

The inventors then embarked on a study to determine whether these findings might be associated with clinical outcome. Taking a small clinical cohort study, the inventors identified mtDNA mutant tumours with >50% VAF of deleterious mtDNA mutations, and surprisingly found that patients with such tumours were 2.5× more likely to respond to nivolumab (anti-PD1) immunotherapy than patients with mtDNA wild-type or low VAF tumours. Specifically, the inventors found that 40% of tumours with >50% VAF responded to this treatment, compared with 17% of <50% VAF tumours responding. Importantly, this study was based on mtDNA samples with a variety of different mutations, which indicates that the inventors' findings are not limited to mutations in the MT-ND5 gene. Merely by way of example, some of the mutations were found in MT-COI, MT-ND4, MT-CYB, MT-TY, and/or the mtDNA control region.

The inventors also found that this difference in treatment responsiveness was valid in the context of treatment with an immune checkpoint inhibitor (such as a PD-1 inhibitor, PD-L1 inhibitor, or CTLA4 inhibitor). Accordingly, the present inventors have identified a novel patient subpopulation that may be particularly susceptible to treatment with an immune checkpoint inhibitor (such as a PD-1 inhibitor, a PD-L1 inhibitor and/or a CTLA4 inhibitor).

Previously, limited efficacy of immune checkpoint inhibitors has been linked to immunosuppressive tumour-associated neutrophils. The inventors have found that treatment responsiveness to an immune checkpoint inhibitor may be further (synergistically) improved in tumours with a high mtDNA mutation load, by co-treatment with compounds that reduce levels of tumour resident neutrophils (such as anti-Ly6G antibodies).

The invention therefore provides a method of predicting whether a subject having a cancer or a pre-cancer would benefit from treatment with an immune checkpoint inhibitor, comprising:
a) determining the deleterious mitochondrial DNA (mtDNA) mutation load in a cancer or pre-cancer sample from the subject; and
b) predicting that the subject would benefit from the treatment when the deleterious mtDNA mutation load is 30% or more.

The invention also provides an immune checkpoint inhibitor for use in treating a cancer or a pre-cancer in a subject, wherein the cancer or pre-cancer has a deleterious mtDNA mutation load of 30% or more.

Further provided is a method of treating a cancer or a pre-cancer in a subject, comprising:
(i) determining the deleterious mtDNA mutation load in a cancer or pre-cancer sample from the subject; and
(ii) administering an immune checkpoint inhibitor if the subject has a deleterious mtDNA mutation load of 30% or more.

Suitably, the immune checkpoint inhibitor may be selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor.

Suitably, the immune checkpoint inhibitor may be selected from the group consisting of a PD-1 inhibitor, a PD-L1 inhibitor, and CTLA4 inhibitor.

Suitably, the deleterious mtDNA mutation load may be 40% or more, 50% or more, or 60% or more.

Suitably, the deleterious mtDNA mutation load may alter the redox status (for example increase the lactate to glucose ratio) in the cancer or pre-cancer to above 3:1.

Suitably, the deleterious mtDNA mutation load may alter the immune micro environment in the cancer or pre-cancer, by:
(i) increasing the level of NK cells;
(ii) decreasing the level of macrophages, optionally wherein the macrophages are in tumour associated macrophages; and/or
(iii) increasing the level of monocytes, optionally wherein the monocytes are immature, intermediate or classical monocytes;
(iv) increasing the level of CD4+ T cells; and/or
(v) increasing the level of ISG-expressing immune cells.

Suitably, the deleterious mtDNA mutation may be selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and/or
(v) a mutation in a mtDNA D-loop region selected from the group consisting of: the H-strand promoter (545-567), MT-HV2 (hypervariable segment 2) m.57-372, and MT-HV1 (hypervariable segment 1)-m. 16024-16390.

Suitably, the deleterious mtDNA mutation may be in a gene selected from the group consisting of: MT-ND5, MT-ND1, MT-ND2, MT-ND3, MT-ND4, MT-ND4L, MT-ND6, MT-CO1, MT-CO2, MT-CO3, MT-CYB, MT-ATP6, MT-ATP8, MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, MT-TY, MT-RNR1 and MT-RNR2.

Suitably, the MT-ND5 deleterious mtDNA mutation may be a truncating mutation that is in a region selected from: m. 12418-12425: A indel or m. 12385-12390: C indel.

Suitably, the deleterious mtDNA mutation may be a truncation, missense, insertion, or frameshift mutation.

Suitably, the cancer or pre-cancer may be selected from the group consisting of: skin, breast, colon, colorectal, oesophageal, thyroid, renal, stomach, ovarian, pancreatic and lung cancer or pre-cancer.

Suitably, the cancer or pre-cancer may be selected from the group consisting of:
(i) a renal cancer or precancer, optionally wherein the renal cancer is a renal cancer papillary and chromophobe subtype;
(ii) a thyroid cancer or pre-cancer, optionally wherein the thyroid cancer is Hürthle cell carcinoma (HCC);
(iii) ovarian cancer or pre-cancer, optionally wherein the ovarian cancer is serous high grade ovarian (SHGO) cancer; and
(iv) colorectal cancer or pre-cancer, optionally wherein the colorectal cancer is colorectal adenocarcinoma.

Suitably, the skin cancer may be melanoma.

Suitably, the PD-1 inhibitor may be nivolumab.

Suitably, the immune checkpoint inhibitor may be for use in combination with a tumour-associated neutrophil reducing compound (such as anti-Ly6G antibody).

The invention also provides a method of predicting whether a subject having a cancer or a pre-cancer would benefit from treatment with an immune checkpoint inhibitor, comprising:
a) determining the redox status (for example lactate to glucose ratio) in the cancer or pre-cancer sample from the subject; and
b) predicting that the subject would benefit from the treatment when there is an altered redox status (for example altered lactate to glucose ratio).

Also provided herein is an immune checkpoint inhibitor for use in treating a cancer or a pre-cancer in a subject, wherein the cancer or pre-cancer has an altered redox status (for example altered lactate to glucose ratio).

The invention also provides a method of treating a cancer or a pre-cancer in a subject, comprising:
(i) determining the redox status (for example lactate to glucose ratio) in a cancer or pre-cancer sample from the subject; and
(ii) administering an immune checkpoint inhibitor if the subject has an altered redox status (for example altered lactate to glucose ratio) in the cancer or pre-cancer.

Suitably, the subject may have an altered redox status (for example altered lactate to glucose ratio) in the cancer or pre-cancer. Suitably, the altered lactate to glucose ratio may be elevated lactate to glucose ratio.

Suitably, the elevated lactate to glucose ratio in the cancer or pre-cancer may be above 3:1.

Suitably, the altered redox status (for example altered lactate to glucose ratio) may alter the immune microenvironment of the cancer or pre-cancer, by:
(i) increasing the level of NK cells;
(ii) decreasing the level of macrophages, optionally wherein the macrophages are in tumour associated macrophages; and/or
(iii) increasing the level of monocytes, optionally wherein the monocytes are immature, intermediate or classical monocytes;
(iv) increasing the level of CD4 NK-like T cells; and/or
(v) increasing the level of ISG-expressing immune cells.

Suitably, the cancer or pre-cancer may be selected from the group consisting of: skin, breast, colon, colorectal, oesophagus, thyroid, renal, stomach, ovaries, pancreas and lung cancer or pre-cancer.

Suitably, the cancer or pre-cancer may be selected from the group consisting of:
(i) a renal cancer or precancer, optionally wherein the renal cancer is a renal cancer papillary and chromophobe subtype;
(ii) a thyroid cancer or pre-cancer, optionally wherein the thyroid cancer is Hürthle cell carcinoma (HCC);
(iii) ovarian cancer or pre-cancer, optionally wherein the ovarian cancer is serous high grade ovarian (SHGO) cancer; and
(iv) colorectal cancer or pre-cancer, optionally wherein the colorectal cancer is colorectal adenocarcinoma.

Suitably, the skin cancer may be melanoma.

Suitably, the PD-1 inhibitor may be nivolumab.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows that mtDNA mutations are abundant in different cancers and provides information on MT-ND5. A—Percentage of well-covered tumours with different types of somatic mtDNA variants per cancer type. Boxes from left to right are: truncating, 2+ types non truncating, rRNA, tRNA, missense, silent, wildtype. Right: number of well-covered samples per cancer type. NSC, non-small-cell cancer. Data from Gorelick et al., 2021. B—Circular mtDNA genome annotated with 73 homopolymer repeat loci ≥5 bp in length. Dot height from the circular mtDNA genome indicates the number of affected samples and dot width indicates the length of the repeat region (5-8 bp). The six solid-colour homopolymer loci highlighted were found to be statistically enriched hotspots for frameshift indels in tumours. Data from Gorelick et al., 2021. C—Space-filling model of respiratory complex I with main reactions/functions annotated. D—Space-filling model of respiratory complex I with internally localised, buried MT-ND5 and its interaction with NDUFB8 protein that is exposed to solvent on both the upper and lower surfaces of complex I highlighted.

FIG. 2 shows how recurrent mutations in tumour Mt-Nd5 were modelled. A—Schematic of mouse mitochondrial genome indicating sites to which DdCBEs are targeted. B—Schematic of TALE DNA binding domains for DdCBE pairs targeted to induce premature stop codons at m. 11,944 and m. 12,436. C—Schematic of TALE-DdCBE library screening method employed. Briefly, candidate pairs were cloned into vectors co-expressing fluorescent marker proteins, allowing sorting of transfected B78 murine melanoma cells by fluorescence-activated cell sorting (FACS). Cells are then assessed for mutagenic efficiency by sequencing.

FIG. 3 shows how recurrent mutations in tumour Mt-Nd5 were generated. A—Heteroplasmy of cells transfected with indicated constructs as determined by pyrosequencing. N transfections indicates cells transfected and recovered once, or four times sequentially. B—mtDNA copy number of cells in FIG. 4A as measured by droplet digital PCR (ddPCR). C—Western blot analysis of marker proteins for the respiratory chain complexes. Complex I (Ndufb8), complex II (Sdhb), complex III (Uqcrc2), complex IV (Mt-Co1) and complex V (Atp5a). Ponceau is shown as a loading control.

FIG. 4 shows how recurrent mutations in tumour Mt-Nd5 were generated. A—Blue native (BN) PAGE and blotting for respiratory chain complexes using antibodies as in FIG. 4C. In gel activity of complex I and complex II following BN PAGE is also shown, along with Coomassie loading control. B—Basal oxygen consumption rate (OCR) of cells as assessed by Seahorse. C—Energy charge state analysis of cells using metabolite abundance data of AMP, ADP and ATP derived from mass spectrometric metabolomic measurements. D—NAD+: NADH ratio as calculated using metabolite abundance data derived from mass spectrometric metabolomic measurements.

FIG. 5 shows the impact of mt-Nd5 mutations on cellular energetics and metabolism. Metabolite abundances derived from mass spectrometric metabolomic measurements of high VAF mutant cells are plotted against each other, revealing consistent metabolic changes due to the two distinct truncating mutations in Mt-Nd5.

FIG. 6 shows that glutamine tracing reveals an increase in MDH1-derived malate abundance in the cytosol. A—heatmap indicating significantly elevated abundance of specific metabolites related to the tricarboxylic acid (TCA) cycle, urea cycle and fumarate adducts. B—schematic of labelling fate for 1-¹³C glutamine. C—abundance of malate m+1. D—abundance of argininosuccinate m+1. E—abundance of a-ketoglutarate (a-KG) m+1. F—abundance of aconitate m+1. G-abundance of aspartate m+1.

FIG. 7 shows that glucose tracing suggests an increase in malate abundance via reverse MDH2 flux. A—schematic of labelling fate for U-¹³C glucose. B—ratio of malate m+3: citrate m+3 derived from mass spectrometric analyses. C—ratio of citrate m+3: aconitate m+3 derived from mass spectrometric analyses. D—ratio of citrate m+3: pyruvate m+3 derived from mass spectrometric analyses.

FIG. 8 shows that MDH1 may mediate the increase in glycolytic intermediates in mutant cells. A—heatmap representation of glycolytic intermediate abundance as detected by mass spectrometry. B—heatmap representation of glycolytic intermediate abundance upon siRNA mediated depletion of MDH1, as detected by mass spectrometry. C—western blot analysis demonstrating knockdown of MDH1 as compared with scrambled siRNA control.

FIG. 9 shows the abundance of specific metabolites in cells treated with siRNA. A—schematic of labelling fate for 4-2H1 glucose. B—malate+1 abundance in cells treated with scrambled siRNA derived from mass spectrometric analyses. C—lactate+1 abundance in cells treated with scrambled siRNA derived from mass spectrometric analyses. D—NADH+1 abundance in cells treated with scrambled siRNA derived from mass spectrometric analyses. E—malate+1 abundance in cells treated with siRNA to MDH1 derived from mass spectrometric analyses. F—lactate+1 abundance in cells treated with siRNA to MDH1 derived from mass spectrometric analyses.

FIG. 10 shows the impact on cancer metabolism in the context of the Krebs cycle (A and B).

FIG. 11 shows the experimental set up used to analyse the situation in vivo.

FIG. 12 shows the impacts in vivo. A—survival curve of mice engrafted with subcutaneous tumours. Humane endpoint is reached when tumours measure 15 mm in any dimension. n=12 mice per genotype. B—dissected tumour weight. n=10-12 tumours per genotype. C—difference in mean heteroplasmy between injected cancer cells and resulting bulk tumour heteroplasmy measurement, as determined by pyrosequencing. n=11-12 per genotype. D—mtDNA copy number analysis of bulk tumour as determined by ddPCR. n=10-12 per genotype. E—metabolite abundances of tumours with indicated genotypes. n=7-10 per genotype.

FIG. 13 shows tumour transcriptional profiling. A—volcano plot presenting differential gene expression between bulk transcriptomics from mtDNA wild-type and 60% VAF m. 11,944 G>A tumours. B—PCA plot of samples compared in A. Each point is a single tumour. C—volcano plot presenting differential gene expression between bulk transcriptomics from mtDNA wild-type and 40% m. 11,944 G>A VAF tumours. D—PCA plot of samples compared in C. Each point is a single tumour. E—volcano plot presenting differential gene expression between bulk transcriptomics from 40% VAF m. 11,944 G>A and 60% VAF m. 11,944 G>A tumours. F—PCA plot of samples compared in E. Each point is a single tumour.

FIG. 14 shows differentially expressed genes; bulk tumour GSEA-wild-type vs VAF >50%. A—summary of differentially expressed genes between mtDNA wild-type and 60% VAF m. 11,944 G>A tumours. B—provides the same information as FIG. 13A. C—Significant hits from gene set enrichment analysis (GSEA) of differentially expressed genes.

FIG. 15 shows differentially expressed genes; bulk tumour GSEA-VAF <50% vs VAF >50%. A—summary of differentially expressed genes between 40% VAF and 60% VAF m. 11,944 G>A tumours. B—provides the same information as FIG. 13C. C—Significant hits from gene set enrichment analysis (GSEA) of differentially expressed genes.

FIG. 16 shows the reshaped immune microenvironment in mtDNA mutant tumours. A—Proportion of natural killer (NK) cells detected in tumours following dissociation and flow cytometry. n=4-8. B—Proportion of tumour-associated macrophages (TAMs) detected in tumours following dissociation and flow cytometry. n=9-14. C—Proportion of immature monocytes detected in tumours following dissociation and flow cytometry. n=10-14. D—Flow cytometry gating strategy for defining TAMs and monocyte populations. E—Flow cytometry gating strategy for defining natural killer cells.

FIG. 17 shows scRNAseq profiling of tumours defines altered immune populations. A—UMAP representation of Seurat clustered single cell RNA sequencing (scRNAseq) data of >100,000 cells harvested from whole, dissociated mtDNA wild-type and 60% VAF m. 12,436G>A tumours. n=3 of each genotype. B—Cell type assignments of scRNAseq data based on CellRanger. C—relative proportions of intermediate monocytes in cluster 3. D—relative proportions of NK cells in cluster 11. E—relative proportions of macrophages in cluster 6. F—relative proportions of classical monocytes in cluster 13. G—relative proportions of interferon stimulatory gene expressing immune cells (ISG Expr. Imm. cells) in cluster 7. H—relative proportions of CD4+NK-like T cells in cluster 24.

FIG. 18 shows scRNAseq profiling of tumours described in FIG. 17. A—GSEA results across all defined clusters for hallmark geneset Interferon gamma response. B—GSEA results across all defined clusters for hallmark geneset Interferon alpha response.

FIG. 19 shows VAF >50% mtDNA mutant melanoma responds to PD1 immune checkpoint blockade. A—schematic of experimental timeline. Cells are engrafted at DO. Once the tumour is palpable, typically at day 7, mice are dosed with intraperitoneal (IP) anti-PD1 monoclonal antibody every 3 days until the experiment concludes at day 21, mice are sacrificed and tumours excised. B—weight of excised tumours from mice bearing tumours with indicated genotype treated with anti-PD1 antibodies as defined in A. n=4-5. C—representative images of excised tumours from B. D—weight of excised tumours from mice bearing tumours with indicates genotype treated with anti-CTLA4 antibodies as defined in A. n=4-7. E—representative images of excised tumours from D.

FIG. 20 shows that this leads to therapeutic sensitivity in humans. A—Stratification of a metastatic melanoma patient cohort by mtDNA status. B—Response rate of patients to nivolumab by tumour mtDNA mutation status. P-values were determined using one-way ANOVA test with Sidak multiple comparisons test (C,F), one-tailed student's t-test (I) or chi-squared test (H) were applied. Error bars indicate SD. Measure of centrality is mean.

FIG. 21 shows representative images of harvested Hcmel12 mutant tumours at day 13 for each drug regimen. Hcmel12 mutant tumours show differential sensitivity to immune checkpoint inhibitors (also referred to herein as immune checkpoint blockage, or ICB).

FIG. 22 shows the tumour weight and growth rate of wild type tumours and complex IV mutated tumours.

FIG. 23 shows the effects of anti-PD1 treatment on wild type tumours and complex IV mutated tumours.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The present disclosure is based on the inventors' identification of a subpopulation of cancer or pre-cancer patients that may be more likely to benefit from treatment with an immune checkpoint inhibitor (such as a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and/or KIR inhibitor). These patients, which the inventors believe are more likely to benefit from such treatment, have a higher deleterious mtDNA mutation load.

The inventors believe that a high deleterious mtDNA mutation load alters the metabolic status of the cancer or precancer cells, as well as the overall tumour microenvironment, which may explain the inventors' finding that different ratios of immune cells are present in tumours comprising such cancer or pre-cancer cells. Specifically, the inventors observed that tumours with a high deleterious mtDNA mutation load have increased numbers of Natural Killer (NK) cells, monocytes, CD4+NK-like T cells, and interferon-stimulated gene (ISG) expressing immune cells, and decreased numbers of macrophages, as compared to tumours with no or low deleterious mtDNA mutation load.

Upon establishing a link between a high deleterious mtDNA mutation load and an altered immune cell population within a tumour, the inventors carried out analysis on patient responsiveness to different immune checkpoint inhibitors. Surprisingly, the inventors found that patients with a high deleterious mtDNA mutation load are more likely to benefit from treatment with an immune checkpoint inhibitor (such as an PD-1 inhibitor, a PD-L1 inhibitor, and/or CTLA-4).

Accordingly, in one aspect, the present invention provides a method of predicting whether a subject having a cancer or a pre-cancer would benefit from treatment with an immune checkpoint inhibitor, comprising:
a) determining the deleterious mitochondrial DNA (mtDNA) mutation load in a cancer or pre-cancer sample from the subject; and
b) predicting that the subject would benefit from the treatment when the deleterious mtDNA mutation load is 30% or more.

In a second, related aspect, the present invention provides an immune checkpoint inhibitor for use in treating a cancer or a pre-cancer in a subject, wherein the cancer or pre-cancer has a deleterious mtDNA mutation load of 30% or more.

As used herein, the term “predicting” refers to assessing the likely reaction of a cancer or pre-cancer in a subject to treatment with an immune checkpoint inhibitor, i.e. assessing the ability of a cancer or pre-cancer to respond favourably to, or to resist, treatment.

If the subject's cancer or pre-cancer is predicted to be likely to respond favourably to the treatment, it can be said that the subject would benefit from the treatment (in other words the treatment is likely or expected to have a therapeutic effect on the subject's cancer or pre-cancer). Such a therapeutic effect may include a clinical improvement of the cancer or pre-cancer in a subject with this disease or condition. A clinical improvement may be demonstrated by an improvement of the pathology and/or symptoms associated with the cancer or pre-cancer. Suitably, therapeutic effect may be demonstrated by preventing the development of the cancer or pre-cancer in a subject, slowing or halting the progression of the cancer or pre-cancer in the subject, or reversing the cancer or pre-cancer. Suitably, the cancer or pre-cancer may be reversed partially, or completely. Clinical improvement of the pathology may be demonstrated by one or more of the following: reduced cancer or pre-cancer biomarker levels in the subject, reduced cancer or pre-cancer cell number in the subject, increased time to regrowth of cancer upon stopping of treatment, prevention or delay of pre-cancer development into cancer, prevention of regrowth of cancer upon stopping treatment, decreased tumour invasiveness, reduction or complete elimination of metastasis, increased cancer cell differentiation, or increased survival rate. Other suitable indications of clinical improvement in the pathology will be known to the skilled person. It will be appreciated that indications of clinical improvement of the pathology will vary depending on the type of cancer. Clinical improvement of symptoms associated with cancer may be, but are not limited to, partial or complete alleviation of pain and/or swelling, increased appetite, reduced weight loss, and/or reduced fatigue.

As used herein, the term “cancer” refers to a large family of diseases which involve abnormal cell growth with the potential to invade or spread to other parts of the body due to the presence of “cancerous cells”. The cancerous cells may form a subset of neoplasms or tumours. A neoplasm or tumour is a group of cells that have undergone unregulated growth, and will often form a mass or lump, but may be distributed diffusely. The tumour or neoplasm may comprise a mixture of cancerous cells (and/or pre-cancerous cells) and healthy (i.e. non-cancerous) cells. The term “tumour” as used herein, encompasses the cancerous and/or pre-cancerous cells, healthy cells (for example stromal cells), as well as the tumour microenvironment which comprises immune cells and the interstitial fluid. The immune cells in the tumour microenvironment may be refers to as the “immune microenvironment” of the tumour.

The term “interstitial fluid” refers to the fluid that occupies the space between the cells (healthy, cancerous, and/or pre-cancers) of the tumour. The interstitial fluid may comprise, metabolites, ions, signalling molecules, proteins, extracellular vesicles, and/or other components secreted by the cells of the tumour and immune cells present therein. As it will be appreciated by the person skilled in the art, a change in the cells of the tumour may lead to change in the interstitial fluid. Merely by way of example, a change in the metabolic status of the cells of the tumour may result in an alteration of the metabolites in the interstitial fluid. As shown by the present inventors, such a change in the metabolic status of the cells of the tumour may alter the tumour microenvironment, for example by altering the immune cell populations within the tumour.

“Cancer cells” may be defined by one or more of the following characteristics: reduced differentiation, self-sufficiency in growth signalling, insensitivity to anti-growth signals, evasion of apoptosis, enabling of a limitless replicative potential, induction and sustainment of angiogenesis, and/or activation of metastasis and invasion of tissue.

A cancer may be a solid cancer or a liquid cancer. Suitably, a cancer may be selected from the group consisting of: a childhood cancer, haematological cancer, and a myeloid cancer. Suitably, a childhood cancer may be selected from the group consisting of: leukaemia, brain cancer, spinal cord cancer, neuroblastoma, Wilms tumour, lymphoma (such as Hodgkin and non-Hodgkin), rhabdomyosarcoma, retinoblastoma, and bone cancer (such as osteosarcoma and Ewing sarcoma). The present application provides examples relating to melanoma. However, the skilled person would appreciate that the aspects of the present invention may apply to other cancers. Nevertheless, the aspects of the present invention may work particularly well in the context of melanoma.

As used herein, “pre-cancer” or a “pre-cancerous condition” is an abnormality that has the potential to become cancer (such a cancer mentioned hereinabove), wherein the potential to become cancer is greater than the potential if the abnormality was not present, i.e., was normal. Examples of pre-cancer include but are not limited to adenomas, hyperplasias, metaplasias, dysplasias, benign neoplasias (benign tumours), premalignant carcinoma in situ, and polyps. In one example, the pre-cancer is a pre-cancer tumour. Such a tumour may comprise pre-cancerous and healthy cells.

As will be clear to a person skilled in the art, the “cancer” and/or “pre-cancer” may be referred to as “a tumour”.

Suitably, in the context of the present disclosure, the cancer or pre-cancer has a deleterious mitochondrial DNA (mtDNA) mutation load.

Suitably, the cancer or pre-cancer may have a high deleterious mitochondrial DNA (mtDNA) mutation load. In the context of the present disclosure, a high deleterious mitochondrial DNA (mtDNA) mutation load may be a mutation load of at least 50% or at least 60%, or more, when determined solely or substantially only on cancer or pre-cancer cells. For example, a high deleterious mitochondrial DNA (mtDNA) mutation load may be a mutation load of at least 70%, at least 80% or more, when determined solely or substantially only on cancer or pre-cancer cells. More suitably, a high deleterious mitochondrial DNA (mtDNA) mutation load may be a mutation load of at least at least 60% when determined solely or substantially only on cancer or pre-cancer cells. Suitably, in the context of the present disclosure, a high deleterious mitochondrial DNA (mtDNA) mutation load may be a mutation load of at least 30%, at least 40%, at least 50% or more, when determined on a sample from the subject. It will be appreciated by a person skilled in the art that a sample will typically comprise a mixture of cancerous cells (and/or pre-cancerous cells) and healthy cells, found within the tumour.

Suitably, the cancer or pre-cancer may have a high nuclear mutation burden. Such a cancer may be referred to as TMB-H (tumour mutation burden-high) cancer. Suitably, the TBM-H cancer may be a solid cancer. Suitably, the solid cancer may be selected from the group consisting of skin cancer (such as melanoma), lung cancer, liver cancer, kidney cancer, and head and neck cancer. Such cancers are generally found to have better sensitivity to immune checkpoint inhibitors, and the present inventors believe that by treating these cancers with agents that alter the redox state (for example the lactate to glucose ratio), the sensitivity to checkpoint inhibitors may be further increased.

In the context of the present disclosure, the term “subject” includes humans and mammals (e.g., mice, rats, pigs, cats, dogs, and horses). In suitable embodiments, subjects are mammals, particularly primates, especially humans. In suitable embodiments, subjects are livestock such as cattle, sheep, goats, cows, swine, and the like; poultry such as chickens, ducks, geese, turkeys, and the like; and domesticated animals particularly pets such as dogs and cats. In certain embodiments (e.g., particularly in research contexts) subject mammals will be, for example, rodents (e.g., mice, rats, hamsters), rabbits, primates, or swine such as inbred pigs and the like. Herein, the terms, “patients” and “subjects” may be used interchangeably.

As mentioned above, the present inventors found that subjects with a high deleterious mtDNA mutation load, that is for example subjects who have a deleterious mtDNA mutation load of 30% or more in cancer or precancer samples obtained from them, are more likely to benefit from treatment with an immune checkpoint inhibitor. Suitably, such subjects have about a 1.25-fold, 1.50-fold, 1.75-fold, 2-fold, 2.25-fold, 2.5-fold, 2.75-fold, 3-fold or more, increased likelihood of an immune checkpoint inhibitor treatment having a therapeutic effect as compared to subjects with no or a low deleterious mtDNA mutation load (wherein “low deleterious mtDNA mutation load may be considered as less than 30% in a cancer or pre-cancer sample, or less than 50% when determined solely or substantially only on cancer or pre-cancer cells).

Immune checkpoint inhibitors are agents that inhibit proteins or peptides (e.g. immune checkpoint proteins) which are blocking the immune system, e.g., from attacking cancer cells. In some examples, the immune checkpoint protein blocking the immune system prevents the production and/or activation of T cells. An immune checkpoint inhibitor can be an antibody or antigen-binding fragment thereof, a protein, a peptide, a small molecule, or combination thereof. Typically, the inhibitor interacts directly to a target immune checkpoint protein (or its ligand, where appropriate) and thereby disrupts its function/biological activity. For example, it may bind directly to a target immune checkpoint protein (or its ligand, where appropriate). In one example, direct binding to a target immune checkpoint protein (or its ligand, where appropriate) inhibits, prevents or reduces the formation of protein complexes which are needed for immune checkpoint protein function/biological activity.

PD-1 inhibitors, PD-L1 inhibitors, and PD-L2 inhibitors are a group of checkpoint inhibitors that block or reduce the activity of PD-1, PD-L1, and PD-L2 immune checkpoint proteins.

A review describing immune checkpoint pathways and the blockade of such pathways with immune checkpoint inhibitor compounds is provided by Pardoll in Nature Reviews Cancer (April 2012). Immune checkpoint inhibitor compounds display anti-tumour activity by blocking one or more of the endogenous immune checkpoint pathways that downregulate an anti-tumour immune response. The inhibition or blockade of an immune checkpoint pathway typically involves inhibiting a checkpoint receptor and ligand interaction with an immune checkpoint inhibitor compound to reduce or eliminate the signal and resulting diminishment of the anti-tumour response.

The immune checkpoint inhibitor compound may inhibit the signaling interaction between an immune checkpoint receptor and the corresponding ligand of the immune checkpoint receptor. The immune checkpoint inhibitor compound can act by blocking activation of the immune checkpoint pathway by inhibition (antagonism) of an immune checkpoint receptor (some examples of receptors include CTLA-4, PD-1, and NKG2A) or by inhibition of a ligand of an immune checkpoint receptor (some examples of ligands include PD-L1 and PD-L2). In such examples, the effect of the immune checkpoint inhibitor compound is to reduce or eliminate down regulation of certain aspects of the immune system anti-tumour response in the tumour microenvironment.

The immune checkpoint receptor programmed death 1 (PD-1) is expressed by activated T-cells upon extended exposure to antigen. Engagement of PD-1 with its known binding ligands, PD-L1 and PD-L2, occurs primarily within the tumour microenvironment and results in downregulation of anti-tumour specific T-cell responses. Both PD-L1 and PD-L2 are known to be expressed on tumour cells. The expression of PD-L1 and PD-L2 on tumours has been correlated with decreased survival outcomes.

Many PD-1 inhibitors and/or PD-L1 inhibitors are known in the art. In some examples, the PD-1 inhibitor and/or PD-L1 inhibitor is a small organic molecule (molecular weight less than 1000 daltons), a peptide, a polypeptide, a protein, an antibody, an antibody fragment, or an antibody derivative. In some embodiments, the inhibitor compound is an antibody. In some embodiments, the antibody is a monoclonal antibody, specifically a human or a humanized monoclonal antibody.

In some examples, the PD-1 inhibitor is an anti-PD-1 antibody or derivative or antigen-binding fragment thereof. In some embodiments, the anti-PD-1 antibody selectively binds a PD-1 protein or fragment thereof. In some embodiments, the anti-PD1 antibody is nivolumab, pembrolizumab, or pidilizumab.

In some examples, the PD-L1 inhibitor is an anti-PDL-1 antibody or derivative or antigen-binding fragment thereof. In some examples, the anti-PD-L1 antibody or derivative or antigen-binding fragment thereof selectively binds a PD-L1 protein or fragment thereof. Examples of anti-PD-L1 antibodies and derivatives and fragments thereof are described in, e.g., WO 01/14556, WO 2007/005874, WO 2009/089149, WO 2011/066389, WO 2012/145493; U.S. Pat. Nos. 8,217,149, 8,779,108; US 2012/0039906, US 2013/0034559, US 2014/0044738, and US 2014/0356353. In some embodiments, the anti-PD-L1 antibody is MEDI4736 (durvalumab), MDPL3280A, 2.7A4, AMP-814, MDX-1105, atezolizumab (MPDL3280A), or BMS-936559.

In some examples, the anti-PD-L1 antibody is MEDI4736, also known as durvalumab. MEDI4736 is an anti-PD-L1 antibody that is selective for a PD-L1 polypeptide and blocks the binding of PD-L1 to the PD-1 and CD80 receptors. MEDI4736 can relieve PD-L1-mediated suppression of human T-cell activation in vitro and can further inhibit tumour growth in a xenograft model via a T-cell dependent mechanism. MEDI4736 is further described in, e.g., U.S. Pat. No. 8,779,108. The fragment crystallizable (Fc) domain of MEDI4736 contains a triple mutation in the constant domain of the IgG1 heavy chain that reduces binding to the complement component C1q and the Fey receptors responsible for mediating antibody-dependent cell-mediated cytotoxicity (ADCC).

CTLA4 inhibitors are inhibitors that block or reduce the activity of CTLA4. The immune checkpoint receptor cytotoxic T-lymphocyte associated antigen 4 (CTLA4 or CTLA-4) is expressed on T-cells and is involved in signaling pathways that reduce the level of T-cell activation. It is believed that CTLA4 can downregulate T-cell activation through competitive binding and sequestration of CD80 and CD86. In addition, CTLA4 has been shown to be involved in enhancing the immunosuppressive activity of TReg cells.

A CTLA4 inhibitor may prevent or reduce binding to CD80 and/or CD86. In some embodiments, a CTLA-4 inhibitor comprises an antibody binding compound, such as an antibody or an antigen-binding fragment thereof. U.S. Pat. Nos. 5,855,887; 5,811,097; 6,682,736; 7,452,535 disclose antibodies specific for human CTLA-4, including antibodies specific for the extracellular domain of CTLA-4 and which are capable of blocking its binding to CD80 or CD86; methods of making such antibodies, and methods of using such antibodies as anti-cancer agents. In some examples, the anti-CTLA-4 antibody is Tremelimumab, Ipilimumab, or Pembrolizumab.

TIGIT (T-cell immunoreceptor containing Ig and ITIM domains) belongs to the immunoglobulin superfamily, also known as Wucam, Vstm3 or Vsig9. TIGIT has an extracellular immunoglobulin domain, type I transmembrane domain and two Immune receptor tyrosine inhibition motif (ITIM). TIGIT is mainly distributed in regulatory T cells (Tregs), activated T cells and natural killer cells (NK), etc. It is a co-suppressive receptor protein, which can be combined with the positive proteins CD226 (Dnam-1) and APC on T cells The expressed ligands CD155 (Pvr or Necl-5) and CD112 (Pvrl-2 or Nectin2) constitute a costimulatory network. Among them, TIGIT competes with CD226 to bind CD155 and CD112, and TIGIT binds its ligand with a higher affinity than CD226. The connection between TIGIT and CD155 or CD112 is mediated by its cytoplasmic ITIM or ITT-like motif, recruiting phosphatase SHIP-1 to the tail of TIGIT to trigger inhibitory signaling. In addition, the ITIM domain is also responsible for the inhibitory ability of mouse TIGIT.

Suitably, TIGIT inhibitors (such as anti-TIGIT antibodies) can inhibit, reduce, or neutralize one or more activities of TIGIT, for example, result in the blocking or reduction of immune checkpoints on T cells or NK cells, or The immune response is reactivated by adjusting antigen presenting cells. Examples of anti-TIGIT antibodies include Vibostolimab, Etigilimab, Tiragolumab, and Domvanalimab.

The term “LAG-3”, “LAG3”, or “Lymphocyte Activation Gene-3” refers to Lymphocyte Activation Gene-3. LAG-3's main ligand is MHC class II, to which it binds with higher affinity than CD4. The protein negatively regulates cellular proliferation, activation, and homeostasis of T cells, in a similar fashion to CTLA-4 and PD-1 and has been reported to play a role in Treg suppressive function. LAG3 is known to be involved in the maturation and activation of dendritic cells. A LAG-3 inhibitor can reduce or block the binding of LAG-3 to the MHC class II molecule, and thereby reduce or block its activity. Suitably, the LAG-3 inhibitor may be an anti-LAG-3 antibody, for example Favezelimab or Relatlimab.

TIM-3 is an immune checkpoint receptor that suppresses antitumor responses by negatively regulating the activity of CD8 T cells and antigen-presenting cells. A TIM-3 inhibitor may reduce or block the activity of TIM-3. Suitably, the TIM-3 inhibitor may be an anti-TIM-3 antibody, for example, Cobolimab.

B and T lymphocyte attenuator (BTLA) is an important co-signaling molecule. It belongs to the CD28 superfamily and is similar to programmed cell death-1 (PD-1) and cytotoxic T lymphocyte associated antigen-4 (CTLA-4) in terms of its structure and function. BTLA can be detected in most lymphocytes and induces immunosuppression by inhibiting B and T cell activation and proliferation. BTLA is found to be expressed in tumor-infiltrating lymphocytes (TILs) and is often associated with impaired anti-tumor immune response. A BTLA inhibitor may reduce or block the activity of BTLA. Such a reduction or blockage may increase B and T cell activation and proliferation. Suitably, the BTLA inhibitor may be an anti-BTLA antibody, for example, Tifcemalimab.

Killer immunoglobulin-like receptors (KIRs), are a family of cell surface proteins found on natural killer (NK) cells. They inhibit the killing function of these cells by interacting with MHC class I molecules. KIR inhibitors may reduce or block the activity of KIR. Such a reduction or blockage may increase the killing ability of NK cells. Suitably, a KIR inhibitor may be an anti-KIR antibody, for example, Lirilumab.

Suitably, the immune checkpoint inhibitor may be selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor.

Suitably, the immune checkpoint inhibitor may be an antibody. For example, the immune checkpoint inhibitor may be an anti-PD-1 antibody, an anti-PD-L1 antibody, an anti-PD-L2 antibody, an anti-CTLA4 antibody, an anti-TIGIT antibody, an anti-LAG-3 antibody, an anti-TIM-3 antibody, an anti-BTLA antibody, and/or anti-KIR antibody.

Monoclonal antibodies, antibody fragments, and antibody derivatives for blocking immune checkpoint pathways can be prepared by any of several methods known to those of ordinary skill in the art, including but not limited to, somatic cell hybridization techniques and hybridoma, methods. Hybridoma generation is described in Antibodies, A Laboratory Manual, Harlow and Lane, 1988, Cold Spring Harbor Publications, New York. Human monoclonal antibodies can be identified and isolated by screening phage display libraries of human immunoglobulin genes by methods described for example in U.S. Pat. Nos. 5,223,409, 5,403,484, 5,571,698, 6,582,915, and 6,593,081. Monoclonal antibodies can be prepared using the general methods described in U.S. Pat. No. 6,331,415 (Cabilly).

As an example, human monoclonal antibodies can be prepared using a XenoMouse™ (Abgenix, Freemont, CA) or hybridomas of B cells from a XenoMouse. A XenoMouse is a murine host having functional human immunoglobulin genes as described in U.S. Pat. No. 6,162,963 (Kucherlapati).

Methods for the preparation and use of immune checkpoint antibodies are well known in the art, and merely by way of example, some are described in the following illustrative publications. The preparation and therapeutic uses of anti-CTLA-4 antibodies are described in U.S. Pat. No. 7,229,628 (Allison), 7311910 (Linsley), and 8017144 (Korman). The preparation and therapeutic uses of anti-PD-1 antibodies are described in U.S. Pat. No. 8,008,449 (Korman) and U.S. Patent Application No. 2011/0271358 (Freeman). The preparation and therapeutic uses of anti-PD-L1 antibodies are described in U.S. Pat. No. 7,943,743 (Korman). The preparation and therapeutic uses of anti-TIM-3 antibodies are described in U.S. Pat. No. 8,101,176 (Kuchroo) and 8552156 (Tagayanagi). The preparation and therapeutic uses of anti-LAG-3 antibodies are described in U.S. Patent Application No. 2011/0150892 (Thudium) and International Publication Number WO2014/008218 (Lonberg). The preparation and therapeutic uses of anti-KIR antibodies are described in U.S. Pat. No. 8,119,775 (Moretta). The preparation of antibodies that block BTLA regulated inhibitory pathways (anti-BTLA antibodies) are described in U.S. Pat. No. 8,563,694 (Mataraza).

In particular examples, the inhibitor of PD1 and/or PD-L1 may be as described in U.S. Pat. No. 8,354,509B2 and U.S. Pat. No. 8,900,587B2 which are incorporated herein by reference. For example, the immune checkpoint therapy is pembrolizumab (also known as KEYTRUDA).

The immune checkpoint inhibitor can be administered in an amount and for a time (e.g., for a particular therapeutic regimen over time) to provide an improvement of the pathology and/or symptoms associated with the cancer or pre-cancer as described herein above.

The immune checkpoint inhibitor may be formulated, dosed, and administered in a fashion consistent with good medical practice. Factors for consideration in this context include, the particular subject being treated, the clinical condition of the individual patient, the cause of the disorder, the site of delivery of the agent, the method of administration, the scheduling of administration, and other factors known to medical practitioners. A “therapeutically effective amount” of an immune checkpoint inhibitor to be administered will be governed by such considerations, and is the minimum amount necessary to prevent, ameliorate, or treat, or stabilize, a benign, precancerous, or early stage cancer; or to treat or prevent the occurrence or recurrence of a tumour, a dormant tumour, or a micrometastases, for example, when used as a neoadjuvant. The immune checkpoint inhibitor need not be, but is optionally, formulated with one or more agents currently used to prevent or treat cancer.

Suitable routes of administration of an immune checkpoint inhibitor include, without limitation, oral, parenteral, subcutaneous, rectal, transmucosal, intestinal administration, intramuscular, intramedullary, intrathecal, direct intraventricular, intravenous, intravitreal, intraperitoneal, intranasal, or intraocular injections. Alternatively, one may administer an immune checkpoint inhibitor in a local rather than systemic manner, for example, via injection of an immune checkpoint inhibitor directly into a solid tumour, or by topical application (for example to a skin cancer).

An immune checkpoint inhibitor may be formulated according to known methods to prepare pharmaceutically useful compositions, whereby the inhibitor is combined in a mixture with a pharmaceutically suitable excipient or carrier. Sterile phosphate-buffered saline is one example of a pharmaceutically suitable excipient. Other suitable excipients are well-known to those in the art. See, for example, Ansel et al, PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Generally, the dosage of an administered immune checkpoint inhibitor for humans will vary depending upon such factors as the patient's age, weight, height, sex, general medical condition and previous medical history. It may be desirable to provide the subject with a dosage that is in the range of from about 1 mg/kg to 24 mg/kg as a single intravenous infusion, although a lower or higher dosage also may be administered as circumstances dictate. A dosage of 1-20 mg/kg for a 70 kg patient, for example, is 70-1,400 mg, or 41-824 mg/m2 for a 1.7-m patient. The dosage may be repeated as needed, for example, once per week for 4-10 weeks, once per week for 8 weeks, or once per week for 4 weeks. It may also be given less frequently, such as every other week for several months, or monthly or quarterly for many months, as needed.

Suitably, the immune checkpoint inhibitor may be employed in the use or method as described herein as a sole treatment for cancer or pre-cancer, or in conjunction with a second treatment for cancer or pre-cancer, such as a surgery, radiation, chemotherapy, immunotherapy, hormone therapy, vaccine treatment, or any combination thereof.

Suitably, the immune checkpoint inhibitor may be employed as first, second, third, or further, line treatment for cancer or pre-precancer.

The method of predicting whether a subject having a cancer or a pre-cancer would benefit from treatment with an immune checkpoint inhibitor, comprises the step of determining the deleterious mitochondrial DNA (mtDNA) mutation load in a cancer or pre-cancer sample from the subject.

Suitably, the immune checkpoint inhibitor (such as a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor) may be for use in combination with a tumour-associated neutrophil reducing compound. A tumour-associated neutrophil reducing compound is a compound that decreases the number of tumour resident neutrophils within a tumour. Herein tumour resident neutrophils may also be referred to as tumour-associated neutrophils. The reduction may be, for example, by blocking tumour resident neutrophil infiltration into the tumour, by reducing the number of neutrophils in the subject (for example by killing and/or blocking the production/maturation of neutrophils), or both. Compounds that may reduce tumour resident neutrophils include for example anti-Ly6G antibody, anti-GR1 antibody, and/or other antibodies that are specific to certain neutrophil antigens (such as antibodies that are specific to the human neutrophil antigens (HNAs), selected from the group consisting of HNA-1a, HNA-1b, and HNA-1c). These antibodies can be used to identify and deplete neutrophils that express these antigens.

The term “decrease” or “decreased” as used herein, generally means a difference between the relevant level (mutation load, number of tumour-associated neutrophils etc.) and a suitable corresponding reference value that is at a reduction of least about 5%, at least about 10%, at least about 20%, at least about 30%, at least about 40%, at least about 50%, at least about 60%, at least about 70%, at least about 80%, at least about 90% etc than the reference value.

In the context of a compound that reduces the number of tumour-associated neutrophils, as used herein, the “reference value” may be the corresponding number of tumour-associated neutrophils in a cancer or a pre-cancer prior to the cancer or pre-cancer being exposed to the compound. Many compounds that reduce the number of tumour-associated neutrophils are known in the art. Additionally, methods of determining the number of tumour-associated neutrophils are known in the art and may be used as a matter of routine.

The tumour-associated neutrophil reducing compound may be used as a pre-treatment. In this context, the agent may be considered as a neoadjuvant. The agent may be provided prior to, or simultaneously with, the immune checkpoint inhibitor (such as PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and/or KIR inhibitor).

The tumour-associated neutrophil reducing compound may be formulated as appropriate. For example, the tumour-associated neutrophil reducing compound may be an infusion. As used herein, “infusion” refers to a solution, emulsion or suspension. In one example, the tumour-associated neutrophil reducing compound may be injected into the cancer or pre-cancer. Typically, the tumour-associated neutrophil reducing compound is agent is a cell permeable compound or a pre-cursor thereof.

The tumour-associated neutrophil reducing compound may be in the form of a pharmaceutical composition. The pharmaceutical composition may further comprise a pharmaceutically acceptable diluent, carrier or excipient. Such compositions may further routinely contain pharmaceutically acceptable concentrations of salt, buffering agents, preservatives, compatible carriers, supplementary immune potentiating agents such as adjuvants and cytokines and optionally other therapeutic agents.

The compositions may also include antioxidants and/or preservatives. As antioxidants may be mentioned thiol derivatives (e.g. thioglycerol, cysteine, acetylcysteine, cystine, dithioerythreitol, dithiothreitol, glutathione), tocopherols, butylated hydroxyanisole, butylated hydroxytoluene, sulfurous acid salts (e.g. sodium sulfate, sodium bisulfite, acetone sodium bisulfite, sodium metabisulfite, sodium sulfite, sodium formaldehyde sulfoxylate, sodium thiosulfate) and nordihydroguaiareticacid. Suitable preservatives may for instance be phenol, chlorobutanol, benzylalcohol, methyl paraben, propyl paraben, benzalkonium chloride and cetylpyridinium chloride.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

It will be appreciated that the pharmaceutical compositions described above may be suitable for use in treating cancer or precancer, and particularly those various forms of cancer described herein.

The tumour-associated neutrophil reducing compound may be for administration to the subject by any suitable route by which a therapeutically effective amount of the agent may be provided.

The terms “mtDNA mutation load”, “mtDNA heteroplasmy”, “variant allele frequency”, or “VAF” refer to mtDNA mutations that arise and co-exist with the wild-type allele in the same cell, or group of cells. In the context of the present disclosure, the term “determine” or “determining” refers to measuring the level of mtDNA molecules comprising a deleterious mutation in a cell or group of cells and comparing that level to the level of mtDNA molecules that do not comprise such deleterious mutations (or to the total number of mtDNA molecules that are present in the cell or group of cells). It will be appreciated that mtDNA molecules that do not comprise deleterious mutations may comprise other mutations, however these mutations would not be deleterious within the meaning of the present disclosure.

MtDNA mutation load may be typically represented as a percentage. For example a mutation load of 30% means that 30% of mtDNA molecules in a cell or group of cells (such as a sample) carry a deleterious mtDNA mutation. The deleterious mutation may be the same or different in all mutated mtDNA molecules. More suitably, the deleterious mutation may be the same in all mutated mtDNA molecules for the purpose of measuring mutation load. However, it will be appreciated that the mtDNA molecules with the deleterious mutation used for determining mutation load may have further (additional) deleterious mutations.

Methods of determining mtDNA mutation loads are well known in the art and include mtDNA sequencing, such as single cell mtDNA sequencing. Methods of determining mtDNA mutation loads are described in, for example, Sobenin et al, 2014 (doi: 10.1155/2014/292017).

In the context of some of the methods disclosed herein, the deleterious mtDNA mutation load is determined in a cancer or pre-cancer sample from the subject.

The term “sample” refers to any group of cells comprising cancer cells and/or pre-cancer cells derived from the subject. The sample may typically comprise a mixture of healthy (i.e. non-cancerous and non-precancerous cells) and cancer cells (and/or pre-cancerous cells). The sample may comprise components of a tumour e.g. cells (cancer, pre-cancer, and healthy cells), as well as interstitial fluid.

Suitably, the sample will comprise at least 5%, at least 10%, at least 15%, at least 20%, or more of cancer and/or pre-cancer cells. For example the sample may comprise at least 25%, at least 30%, at least 35%, at least 40%, at least 45%, at least 50% or more of cancer and/or pre-cancer cells. The presence of healthy cells, which may be substantially free of a deleterious mtDNA mutation load, may lower the determined (overall) deleterious mtDNA mutation load in a sample as compared to if the deleterious mtDNA mutation load was determined solely or substantially only on cancer or pre-cancer cells. In this context the term “substantially only” means that the cancer cells account for at least 50%, at least 60%, at least 70%, at least 80%, at least 90%, or more of the cells in the sample. Thus, merely by way of example, when a deleterious mtDNA mutation load in a cancer or pre-cancer sample obtained from a subject is determined to be about 30%, the deleterious mtDNA mutation load of the cancer or precancer cells present in the sample specifically may be more than 30%, more than 40%, more than 50%, more than 60%, more than 70%, more than 80%, or more than 90%.

Suitably, with reference to a solid cancer, the sample may be a biopsy, a smear sample, or a interstitial fluid sample.

Suitably, with reference to a liquid cancer, the sample may be a blood sample (for example, a whole blood sample, a blood plasma sample, or a serum sample), or a urine sample.

The term “deleterious mtDNA mutation” as used herein refers to a mutation that adversely affects the structure and/or function of the mtDNA element it encodes, in contrast to a neutral mutation (such as a silent point mutation), which has neither a positive or negative mutation on the corresponding encoded element.

Methods of identifying deleterious mtDNA mutations will be known in the art. Merely by way of example, a deleterious mtDNA mutation may be selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and/or
(v) a mutation in a mtDNA D-loop region selected from the group consisting of: the H-strand promoter (m. 545-567), hypervariable segment 2 (MT-HV2; m.57-372), and hypervariable segment 1 (MT-HV1; m. 16024-16390).

The tRNA mutation may be in a gene selected from the group consisting of MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, and MT-TY.

The rRNA mutation may be in a gene selected from the group of MT-RNR1 and MT-RNR2.

The truncation or missense mutation may be in a tRNA, rRNA or protein coding gene.

The protein coding gene may be selected from the group of MT-ND5, MT-ND1, MT-ND2, MT-ND3, MT-ND4, MT-ND4L, MT-ND6, MT-CO1, MT-CO2, MT-CO3, MT-CYB, MT-ATP6, and MT-ATP8. These genes encode proteins that are subunits of the mitochondrial respiratory chain complexes, specifically NADH: ubiquinone oxidoreductase (complex I), ubiquinol: cytochrome c oxidoreductase (complex III), cytochrome c oxidase (complex IV), or ATP synthase (complex V). Accordingly, the mutation may be in a mtDNA gene that encodes a subunit of a mitochondrial respiratory chain complex selected from the group consisting of complex I, complex III, complex IV and complex V.

Suitably, the deleterious mtDNA mutation is a truncation, missense, insertion, or frameshift mutation.

Suitably the deleterious mutation may be in the gene MT-ND5. Suitably, the deleterious mutation may be a truncating mutation that is in a region selected from: m. 12418-12425: A indel or m. 12385-12390: C indel.

Suitably, the deleterious mutation may be a missense mutation in the MT-CO1, MT-ND5, MT-ND4, MT-CYB or MT-TY gene.

Suitably, the missense mutation may be selected from the group consisting of m.6318C>T, m. 12730G>A, m. 11736T>C, m. 15140G>A, and m.5843A>G.

Suitably, the insertion mutation may be selected from the group consisting of m. 16183: CC indel, and m. 16192: T indel.

As will be clear to the person skilled in the art, the amount of mtDNA molecules having the deleterious mutation is used to determine the level of the mutation load in a cell or a group of cells. Herein, the proportion of mtDNA molecules having the deleterious mutation is referred to as “the deleterious mtDNA mutation load”. The skilled person would appreciate that a low proportion of mtDNA molecules having the deleterious mutation will correspond to a low deleterious mtDNA mutation load, which may be asymptomatic (i.e. have little or no impact on overall mitochondrial function of the cell). Conversely, a high proportion of mtDNA molecules having the deleterious mutation will correspond to a high deleterious mtDNA mutation load, which in the context of the present disclosure may be symptomatic, i.e. have an adverse effect on overall mitochondrial function of the cell. An adverse effect on overall mitochondrial function of the cell may be determined by an altered redox status, which may be due to, for example: altered mitochondrial redox homeostasis, reduced oxidative phosphorylation, increased oxidative stress, or any combination thereof. These changes may further lead to alterations in the cancer or pre-cancer microenvironment, such as the tumour as a whole and/or interstitial fluid of the cancer or pre-cancer (also referred to herein as the interstitial fluid of the tumour). The altered tumour microenvironment may be more or less favourable for specific immune cell populations, as explained in more detail hereinbelow.

The term “redox status” or “metabolic status” as used herein refers to the cytosolic and/or mitochondrial ratio of NAD+:NADH in the cancer or pre-cancer microenvironment, such as the tumour as a whole and/or interstitial fluid of the cancer or pre-cancer (also referred to herein as the interstitial fluid of the tumour). The inventors have found that both decreasing the NAD+:NADH ratio (mtDNA mutation) and/or increasing the NAD+:NADH ratio away from homeostatic levels within cancer and/or pre-cancer cells exerts an immunomodulatory effect on tumours, rendering these more sensitive to immune checkpoint inhibitors. Homeostatic levels in this context may refer to the levels in wild-type (for example non-cancerous cells, and/or cancer cells that do not bear mtDNA mutations). NAD+:NADH ratio is tightly regulated in cells—as the directionality and activity of a huge number of reactions (glycolysis, gluconeogenesis, fatty acid synthesis, DNA repair (PARP is NAD+dependent) histone acetylation etc) are dependent on it. An altered redox status may be indicated by an increase in one or more cellular metabolite selected from the group consisting of: fumarate, lactate, malate, acetyl COA, aspartate, glucose, glucose 6-phosphate, glutamine, glucose 3-phosphate, glycolytic intermediates, and fumarate adducts (such as succinicGSH and/or succinylCysteine). In particular, an altered redox status may be indicated by an increase in the fumarate adducts succinicGSH and/or succinylCysteine (also referred to as succ.cys and succ.gsh respectively herein).

Additionally, or alternatively, altered redox status may be indicated by a decrease in one or more cellular metabolite selected from the group consisting of: alpha-ketoglutarate, pyruvate, phosphoenolpyruvate and succinate.

A deleterious mtDNA mutation load may alter the NAD+:NADH ratio in the mitochondria and/or the cytosol. Suitably, the deleterious mtDNA mutation load may increase the NAD+:NADH ratio in the mitochondria and/or the cytosol. As shown in the examples, disturbed NAD+:NADH ratio may result in partial reverse flux of MDH2 within mitochondria (which can be determined from the ratio of pyruvate carboxylase-derived (m+3) malate, citrate, and aconitate and pyruvate).

Merely by way of example, altered redox status may include changes in TCA cycle and/or urea cycle metabolites. Suitably, these metabolites may be related to the malate-aspartate shuttle (MAS) and fumarate within mitochondria and/or within the cytosol. As described in detail in the examples, the inventors used 1-¹³C-glutamine tracing, which revealed that NAD+:NADH ratio changes are associated with increases in malate m+1 abundance, and argininosuccinate m+1 abundance, but not a-KG m+1, aconitate m+1 or aspartate m+1-implicating increased MDH1 flux. Accordingly, altered mitochondrial metabolic state may include increased MDH1 flux.

Merely by way of example, altered redox status may include an imbalance between lactate and glucose in the tumour (e.g. in the interstitial fluid of the tumour). Accordingly, a deleterious mtDNA mutation load may alter the redox status (for example alter the lactate to glucose ratio) in the tumour (e.g. in the interstitial fluid of the tumour). Suitably, the deleterious mtDNA mutation load may increase the lactate to glucose ratio in the tumour (e.g. in the interstitial fluid of the tumour) to above 2.5:1, 3:1, 3.5:1, 4:1 or more.

Merely by way of example, altered redox status may include an imbalance between pyruvate and lactate in the tumour (e.g. in the interstitial fluid of the tumour). Accordingly, a deleterious mtDNA mutation load may alter the redox status (for example alter the pyruvate to lactate ratio) in the tumour (e.g. in the interstitial fluid of the tumour).

The term “altered” or “imbalance” as used herein refers to a change, which may be an increase or a decrease, relative to a reference value.

The term “increased” or “increase” as used herein generally means a difference between the relevant level (mutation load, metabolite etc) and a suitable corresponding reference value, that is at least about 10% greater than the reference value, for example at least about 20%, at least about 30%, at least about 40%, at least about 50%, at least about 60%, at least about 70%, at least about 80%, at least about 90% greater than the reference value.

The term “decrease” or “decreased” as used herein, generally means a difference between the relevant level (mutation load, metabolite etc) and a suitable corresponding reference value that is at a reduction of least about 5%, at least about 10%, at least about 20%, at least about 30%, at least about 40%, at least about 50%, at least about 60%, at least about 70%, at least about 80%, at least about 90% etc than the reference value.

As used herein, the “reference value” may be derived from a corresponding sample from a healthy subject or subject that has a cancer or pre-cancer having no or substantially no deleterious mtDNA mutation load. It may be derived from a healthy sample of a subject that has a cancer or pre-cancer. The reference value may be derived from the same subject or a different subject. Suitably, the reference value may be derived from a single subject (or a sample from a single subject) or may be derived from a group of subjects (or a group of samples).

The present inventors have shown that the presence of a deleterious mtDNA mutation load in cancers and precancers results in the redox status (for example lactate to glucose ratio) in the cancer or pre-cancer being altered. The inventors believe that this may be the reason why such cancers or pre-cancers have a notably different proportion of immune cells (as compared to cancers with low or substantially no deleterious mtDNA mutation load). As shown in the Examples section herein, the inventors have found that cancers with a high deleterious mtDNA mutation load are associated with increased levels of immune cells selected from the group consisting of: NK cells; monocytes; CD4 NK-like T cells; and ISG-expressing immune cells.

The term “NK cells” or “Natural Killer cells” as used herein refers to a subset of peripheral blood lymphocytes defined by the expression of CD56 or CD16 and the absence of the T cell receptor (CD3).

The term “monocytes” as used herein refers to a subset of immune cells that are produced in the bone marrow and migrate through the blood to tissues in the body, where they become a macrophage. Suitably the monocytes are immature, intermediate or classical monocytes. Immature monocytes are Lys6C and F480 positive. Intermediate monocytes are CD14+ and CD16+. Classical monocytes are CD14+ and CD16−.

The term “CD4 NK-like T cells” as used herein refers to a subset of immune cells that are cytotoxic T-cells that co-express NK receptors such as CD56, CD16, and/or CD57.

The term “ISG-expressing immune cells” refers to a subset of cells that express interferon-stimulated genes.

Furthermore, the inventors also found that cancers with a high deleterious mtDNA mutation load have decreased levels of macrophages (for example tumour associated macrophages).

The term “macrophages” refers to a subgroup of phagocytic cells produced by monocyte differentiation. The term “tumour associated macrophages” (TAMs) generally refers to macrophages that exist in the microenvironment of a cancer, for example, a tumour.

Suitably, NK cell levels may be increased by at least at least 100%, at least 150%, at least 200% etc. Suitably, the levels of tumour associated macrophages may be decreased by at least 25%, at least 50%, at least 75% etc. Suitably, the levels of immature monocytes may be increased by at least 100%, at least 150%, at least 200% etc.

The above mentioned changes in the cancer or pre-cancer immune microenvironment have been shown herein to be an accurate means for predicting that the cancer or pre-cancer would benefit from treatment with an immune checkpoint inhibitor (such as PD-1 inhibitor, a PD-L1 inhibitor, and/or CTLA-4 inhibitor). The present inventors believe that this may be true regardless of whether the cancer or pre-cancer also has a high deleterious mtDNA mutation load. This gives rise to the following aspects of the present invention.

A method of predicting whether a subject having a cancer or a pre-cancer would benefit from treatment with an immune checkpoint inhibitor is provided herein, comprising:

a) determining the redox status (for example lactate to glucose ratio) in a cancer or pre-cancer sample from the subject (e.g. determining the redox status, such as lactate to glucose ratio in the interstitial fluid of a cancer or pre-cancer sample from the subject); and b) predicting that the subject would benefit from the treatment when there is an altered redox status (for example altered lactate to glucose ratio).

Provided herein is also an immune checkpoint inhibitor for use in treating a cancer or a pre-cancer in a subject, wherein the cancer or pre-cancer has an altered redox status (for example altered lactate to glucose ratio) (e.g, wherein the cancer or pre-cancer has an altered redox status, such as lactate to glucose ratio in the interstitial fluid).

Further provided herein is a method of treating a cancer or a pre-cancer in a subject, comprising:
(i) determining the redox status (for example lactate to glucose ratio) in a cancer or pre-cancer sample from the subject (e.g. determining the redox status, such as lactate to glucose ratio in the interstitial fluid of a cancer or pre-cancer sample from the subject); and
(ii) administering an immune checkpoint inhibitor if the subject has an altered redox status (for example an altered lactate to glucose ratio).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1

### Results

The inventors have performed the experiments detailed below. The data that has been generated is shown in the corresponding Figures and is also described in the corresponding Figure legends.

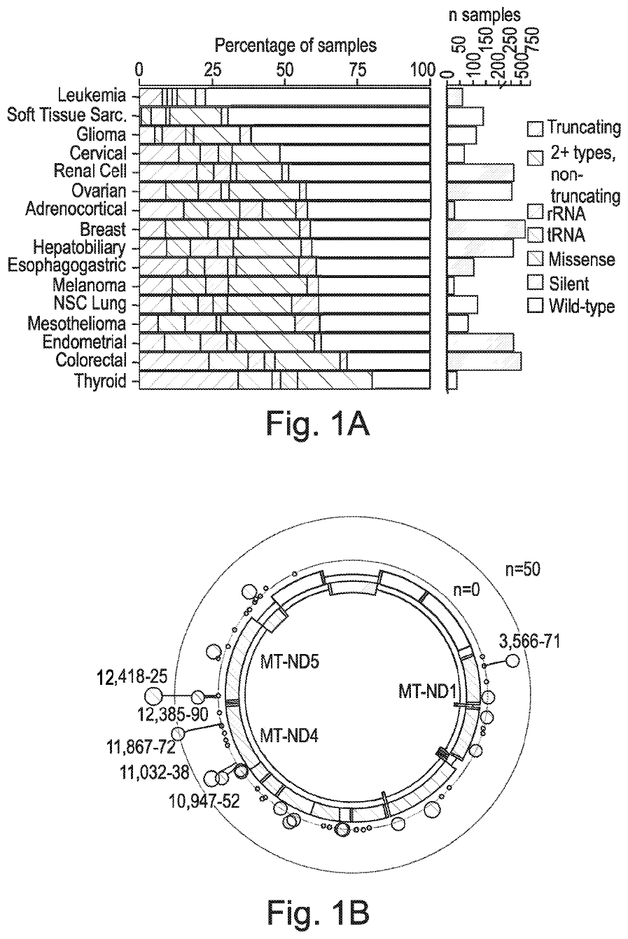

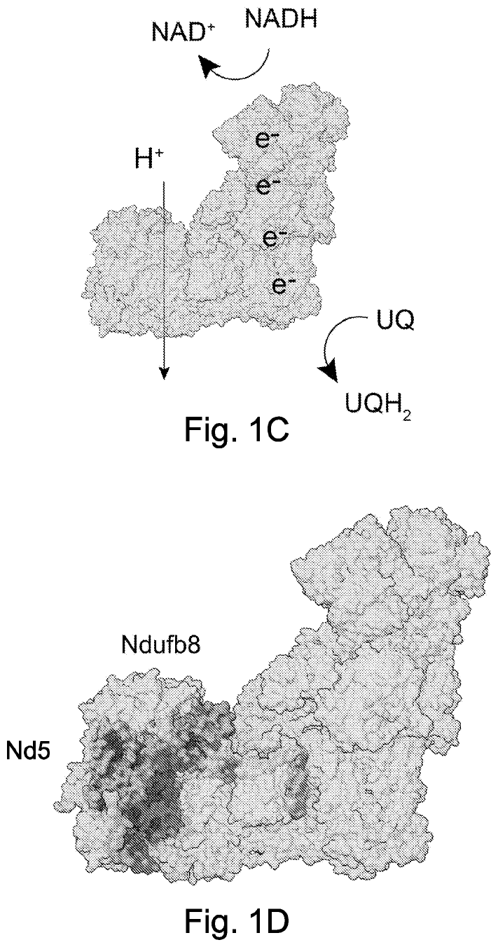

The inventors have previously identified that mtDNA mutations are abundant in cancer (see FIG. 1 herein, obtained from data in Gorelick et al., 2021). Interestingly, they observed high levels of truncating mutations with recurrence at specific positions in mtDNA that have not previously been observed. The majority of these are in complex I genes (MT-ND)—with ND5 being the most commonly impacted. Complex I is the part of the respiratory chain, oxidizing NADH to NAD+ and transferring these electrons to ubiquinone (Q) in a two electron reduction to produce ubiquinol (QH2), the energy of which is coupled to pumping proton across the mitochondrial inner membrane.

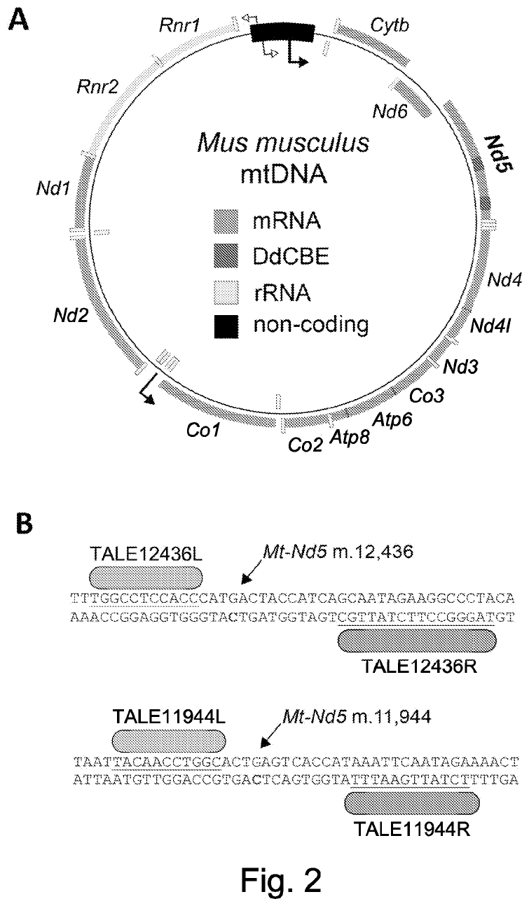

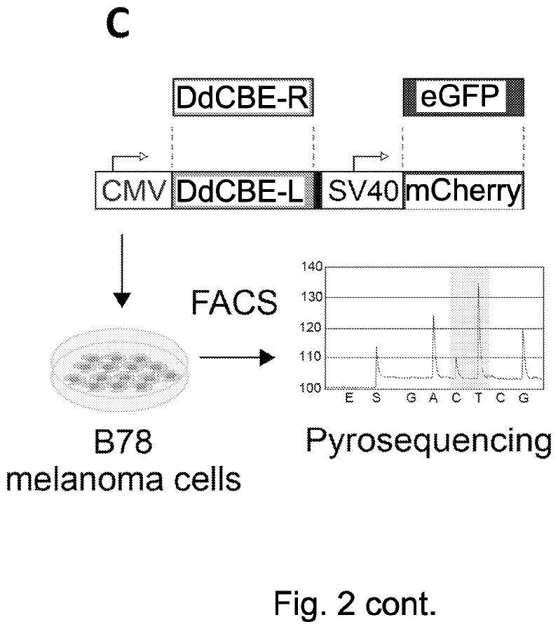

To study the role of truncating mutations in ND5, the inventors have now designed DdCBEs (mitochondrial base editing enzymes) to regions in ND5 where premature stop codons could conceivably be introduced (see FIG. 2). After screening a library of candidates in B78 melanoma cells, the inventors identified reagents which efficiently mutated m. 12,436 G>A and m. 11,944 G>A in Mt-Nd5, both of which convert a tryptophan codon into an in frame stop codon (TGA >TAA).

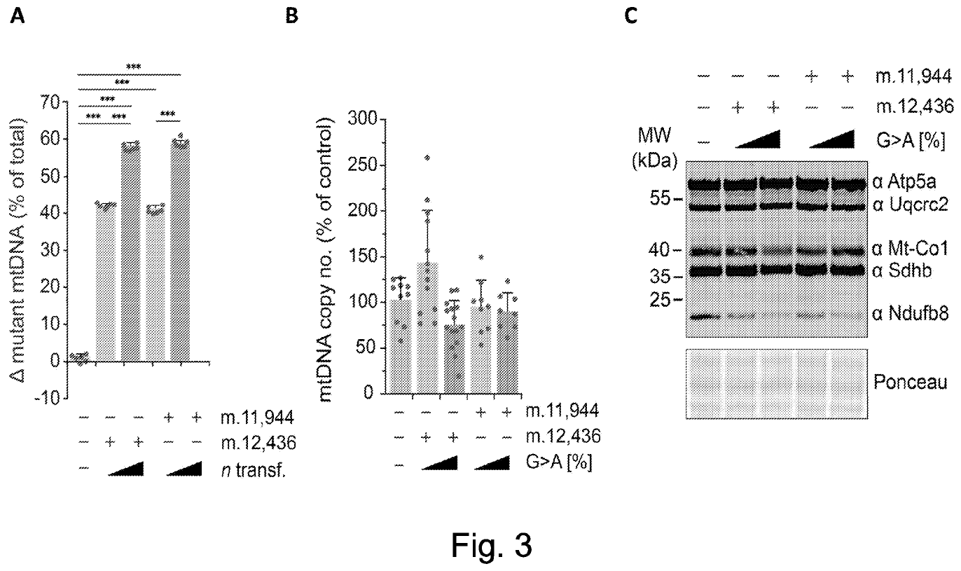

Using these reagents the inventors were able to produce cell lines with either a ˜40% or a ˜60% heteroplasmy (or variant allele frequency, VAF) of both of the truncating mutations (see FIG. 3). These mutations did not impact mtDNA copy number, however a mutation dose-dependent loss of Ndufb8 protein expression was observed, as would be predicted if lower levels of Mt-Nd5 were produced.

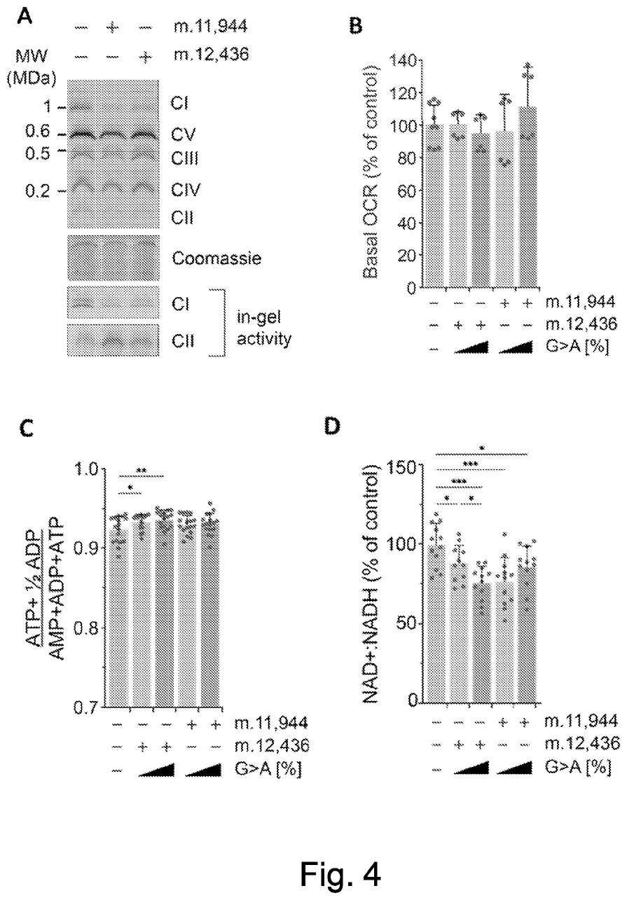

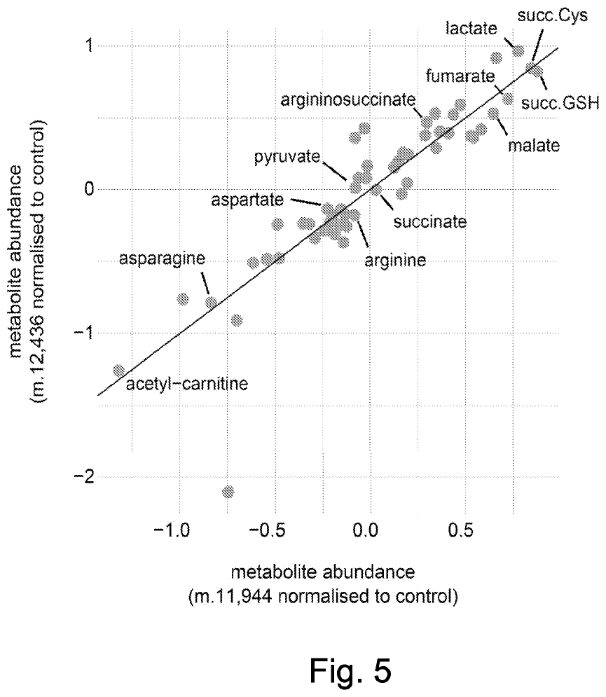

Using native gels the inventors determined that intact complex I levels in 60% VAF cells were diminished in comparison with parental cells (see FIG. 4). However, oxygen consumption (OCR) was unaffected, and adenylate charge was also not negatively impacted, suggesting that these cells are not in energetic crisis. Importantly, these cells demonstrated an altered NAD+:NADH ratio, which is to be expected as complex I is a major cellular site of NADH oxidation. A comparison of 60% VAF cells using metabolomic profiling was carried out and revealed equivalent changes in metabolic profiling between the two genetically distinct models of Mt-Nd5 truncation (see FIG. 5).

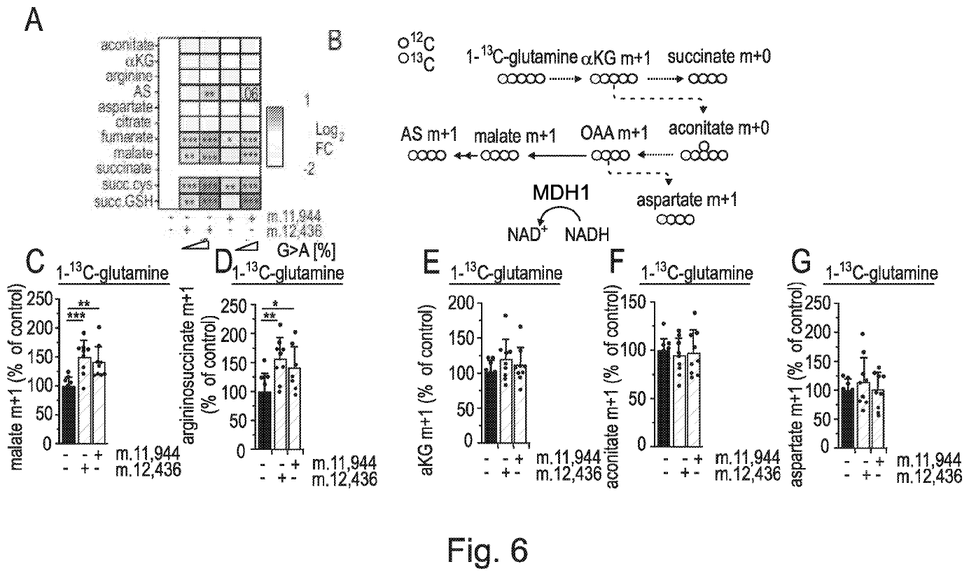

Changes in TCA cycle metabolites potentially related to the cytosolic component of the malate-aspartate shuttle (MAS) and fumarate handling led the inventors to assess the components of the MAS within mitochondria as well as assessing these components within the cytosol (see FIG. 6). To study the MAS from the cytosol, the inventors used 1-¹³C-glutamine tracing, which revealed that NAD+:NADH ratio changes are associated with increases in malate m+1 abundance, and argininosuccinate m+1 abundance, but not a-KG m+1, aconitate m+1 or aspartate m+1-implicating increased MDH1 flux rather than a) increased reductive carboxylation of glutamine or b) increased generation of arginosuccinate (then subsequently fumarate and malate) from aspartate that contributes to increased steady-state abundance of malate.

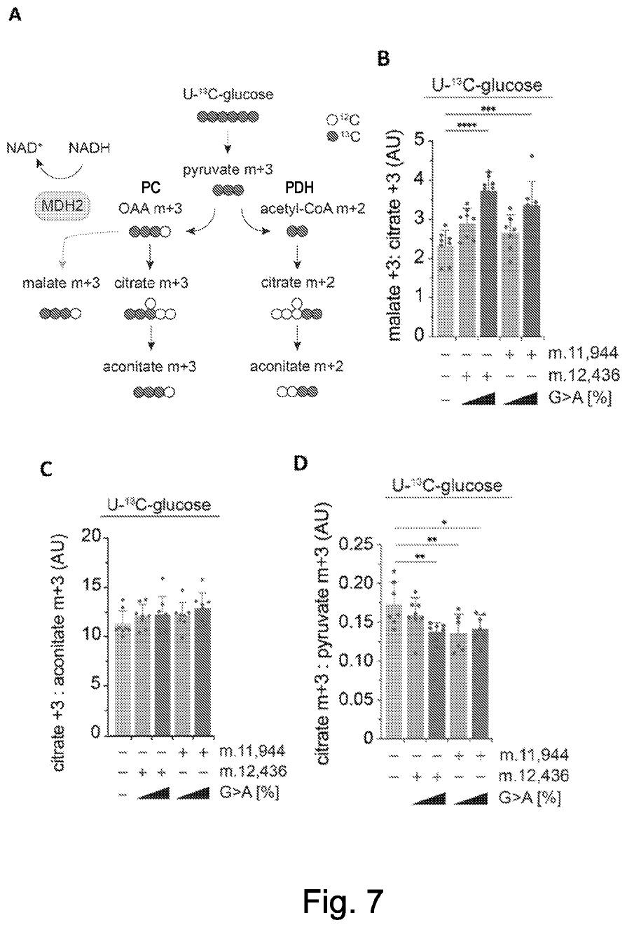

Changes in TCA cycle metabolites potentially related to the cytosolic component of the malate-aspartate shuttle (MAS) and fumarate handling led the inventors to assess the components of the MAS from within mitochondria as well as assessing these components within the cytosol. Using U-¹³C-glucose the results indicated that disturbed NAD+:NADH ratio results in partial reverse flux of MDH2 within mitochondria, determined from the ratio of pyruvate carboxylase-derived (m+3) malate, citrate, aconitate and pyruvate (see FIG. 7).

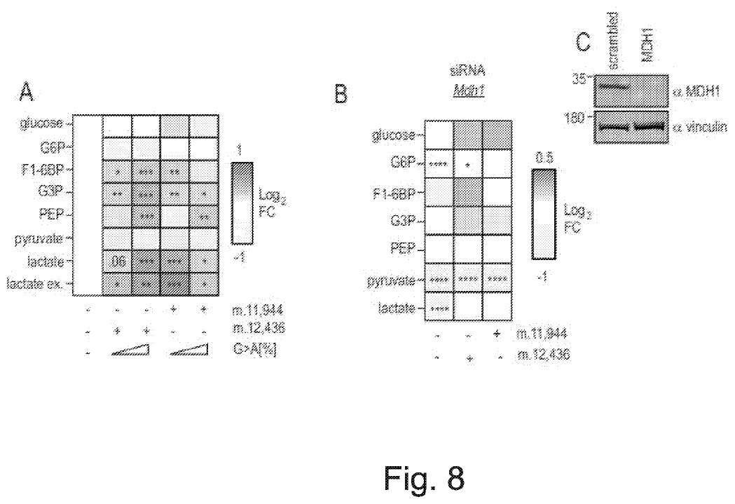

ND5 mutations in these cells are also associated with increased abundance of glycolytic intermediates (see FIG. 8, particularly FIG. 8A). MDH1 has previously been described to facilitate (by unknown means but likely physical interaction) NADH shuttling between GAPDH and MDH1. The inventors suspected that elevated cellular NADH could conceivably be counterbalanced by MDH1 regenerating NAD+ through enhanced oxidation of glucose and via the interaction with GAPDH. When Mdh1 is knocked down using siRNA (see FIG. 8B), substantial changes in glycolytic intermediate abundances are observed, further implicating NAD+:NADH imbalance-driven MDH1 activity in supporting the enhanced glycolytic intermediate abundances seen in FIG. 8A.

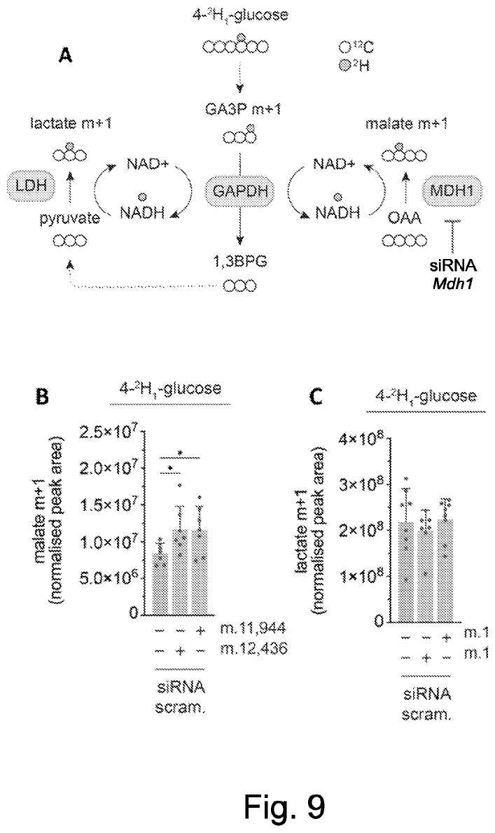

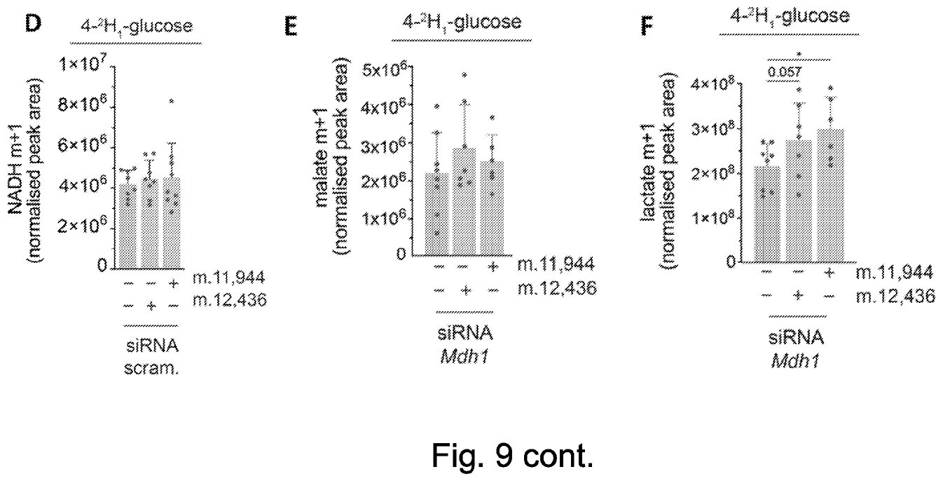

Using 4-²H₁ glucose tracing the inventors demonstrated that there is preferential shuttling of electrons from glucose to malate in Mt-Nd5 mutant cells, which is not due to increased levels of cellular NADH m+1 (see FIG. 9). Lactate m+1 is unaffected. This preferential shuttling is abolished upon knockdown of MDH1. Knockdown of MDH1 is also associated with decreased glycolytic flux (as shown in FIG. 8) and proportionally increased labelling of lactate +1 in ND5 mutant cells, suggesting that GAPDH may also shuttle electrons to LDH in specific circumstances.

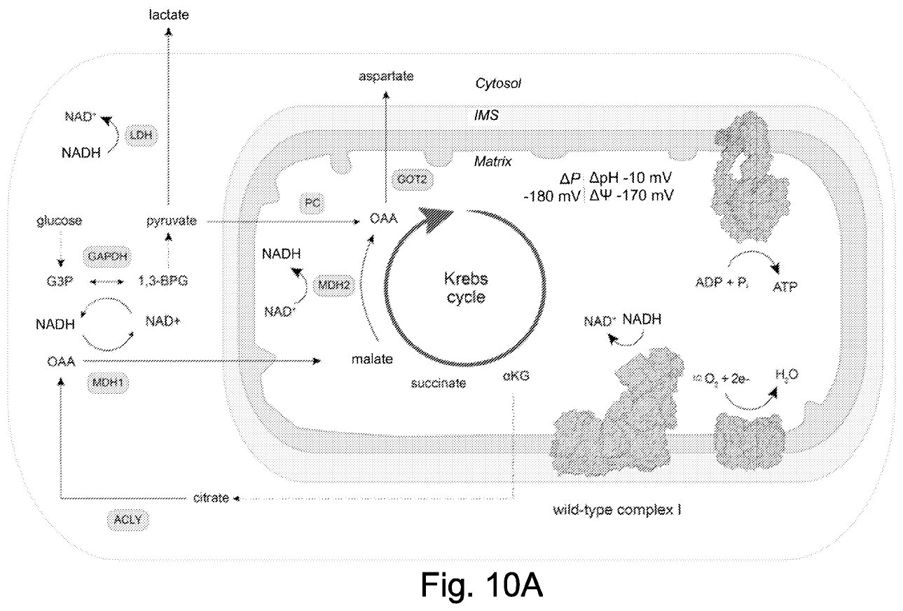

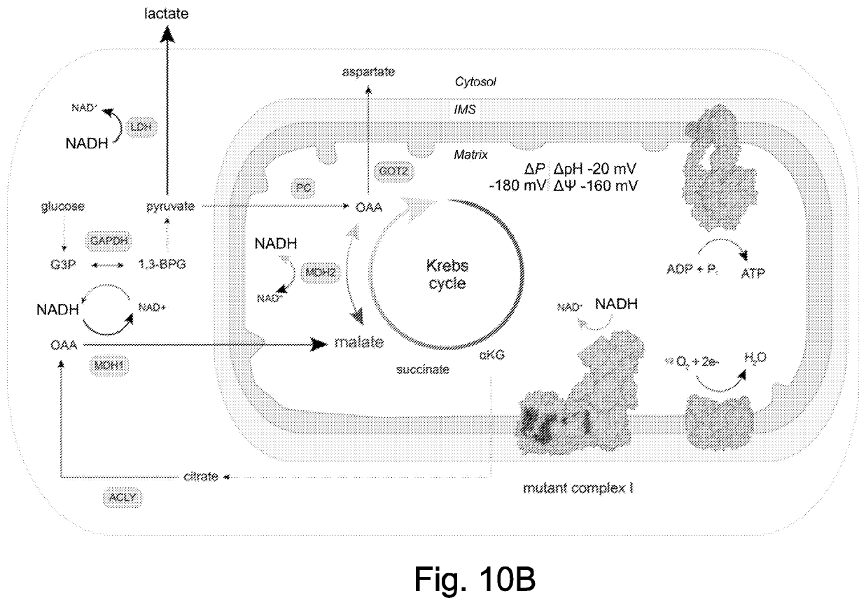

FIG. 10A shows a summary of the relevant metabolic pathways. A summary of how these change when Mt-Nd5 truncations are present is shown in FIG. 10B. Briefly, high NADH results in reverse flux of MDH2 and accumulation of cytosolically produced malate via MDH1. This results in elevated fumarate which results in increased argininosuccinate synthesis and production of fumarate adducts succinicGSH/succinylCysteine (not shown in schematic for clarity). The increased MDH1 activity drives glycolysis and results in excess glucose consumption/lactate release. Oxygen consumption and ATP synthesis remain unaffected at 60% VAF, although they are likely to be impacted at higher VAF.

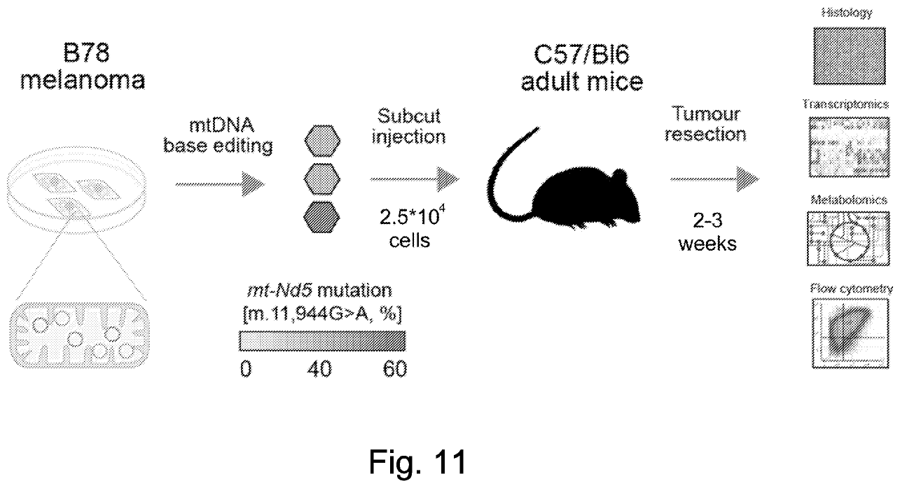

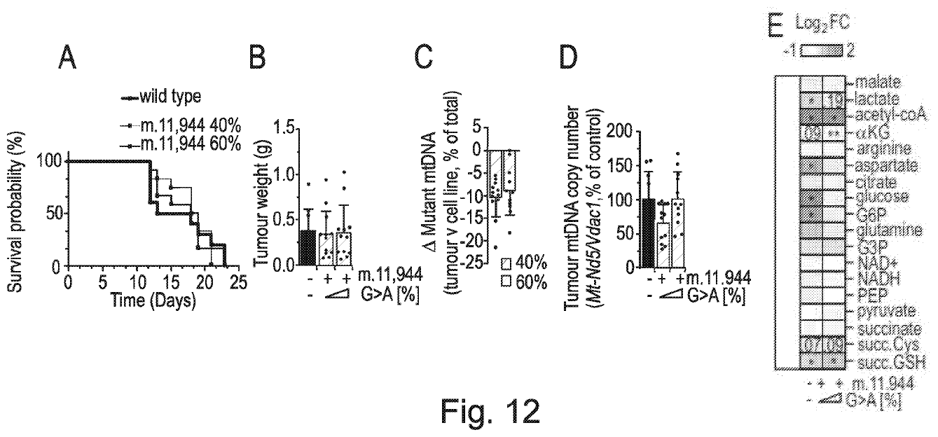

The inventors then characterized murine melanoma cells in vivo by implanting them into immunocompetent mice and allowing tumours to form (see experimental plan in FIG. 11). No gross differences in time to endpoint or tumour weight were observed, between wildtype (WT) and mutant, or between different VAFs. The difference in VAF between the implanted cells and the resulting tumours (estimated from bulk DNA extraction) is not VAF dependent and doesn't show clear selection (downward trend likely due to stromal contamination), and there is no observable difference in mtDNA copy number of the tumours either, however there are clear metabolic changes between both low and high VAF tumours and control (see FIG. 12). Of particular note are succ. Cys and succ. GSH.

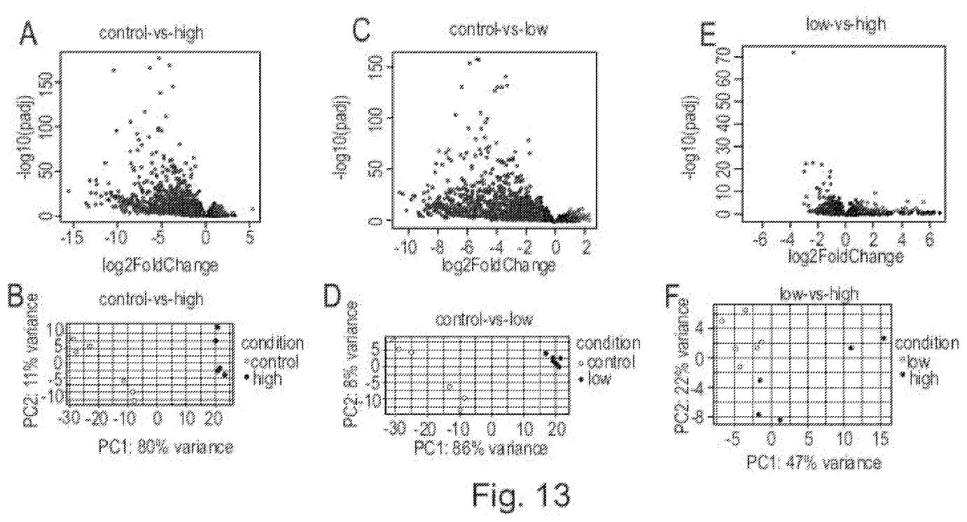

While the tumours do not appear to be different by measures used so far, when examined by bulk RNAseq there are clear and substantial differences in transcriptional profiles between control and high VAF, and control and low VAF tumours. There are more modest, but still some significant changes, between low VAF and high VAF tumours (see FIG. 13).

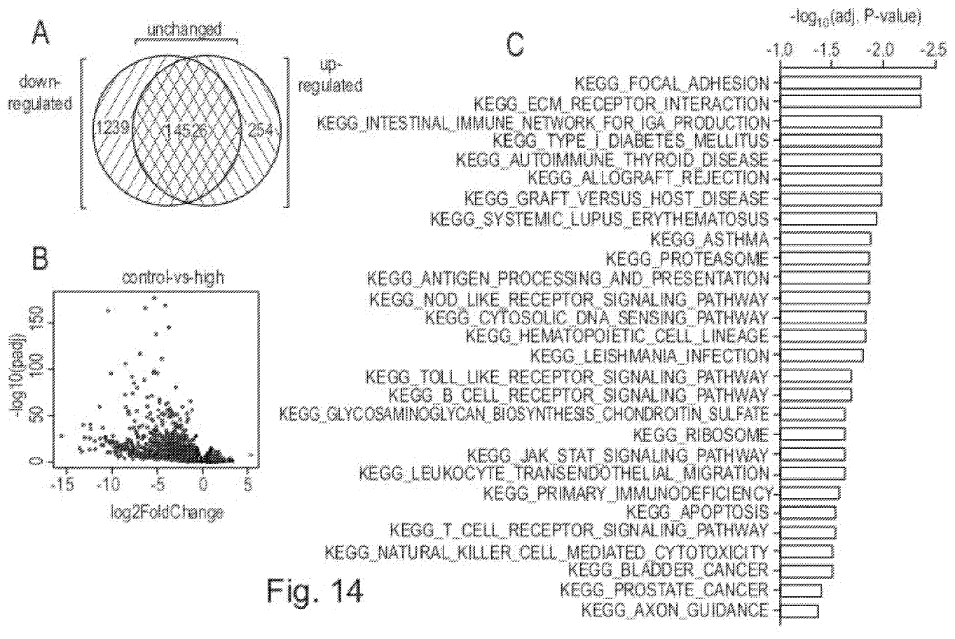

Comparing control and high VAF transcriptional data using geneset enrichment analysis revealed a host of processes that are differentially regulated, mainly relating to cell-cell interactions, receptor signalling and the immune system (see FIG. 14). Of particular note, Natural Killer Cell Mediated Cytotoxicity.

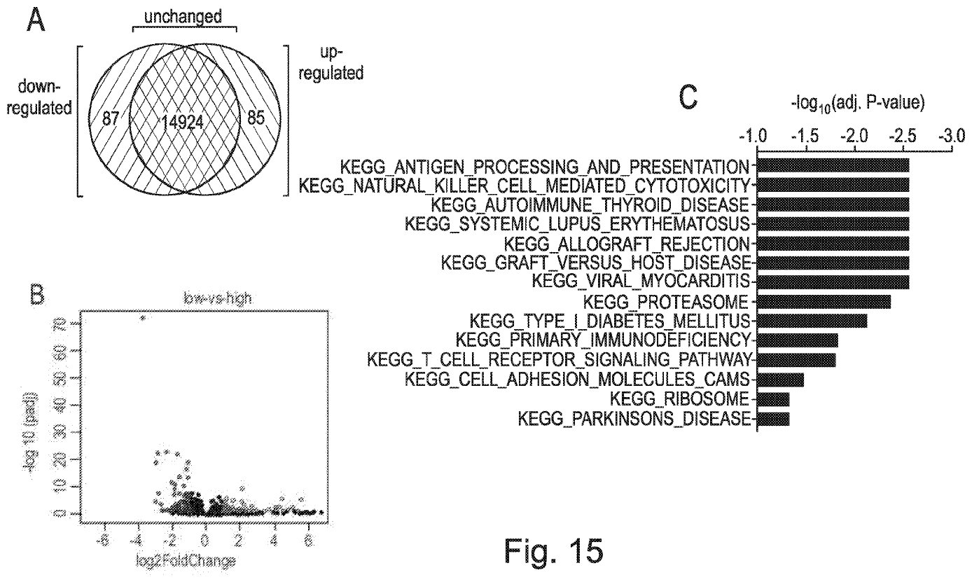

When comparing low and high VAF tumours, the list of significantly altered GSEA outputs is much shorter, and is now focused more broadly on the immune system (see FIG. 15). Again, of particular note is Natural Killer Cell Mediated Cytotoxicity.

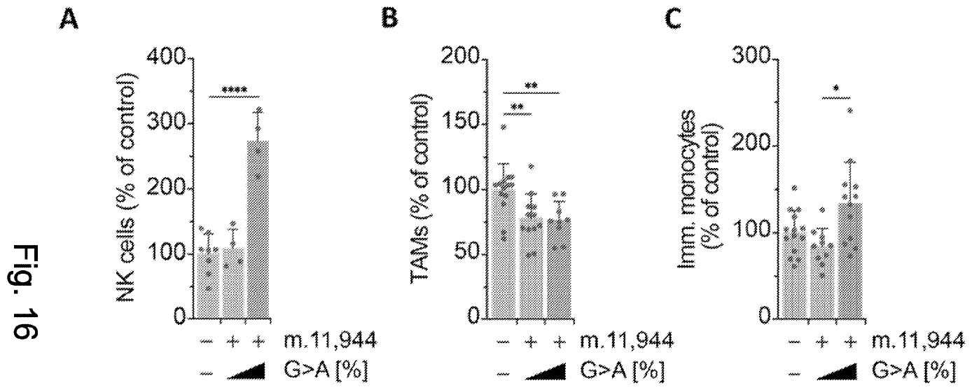

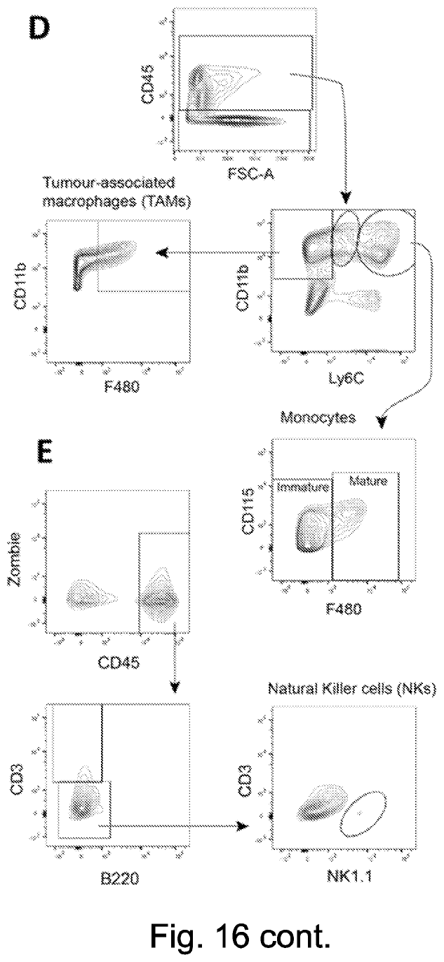

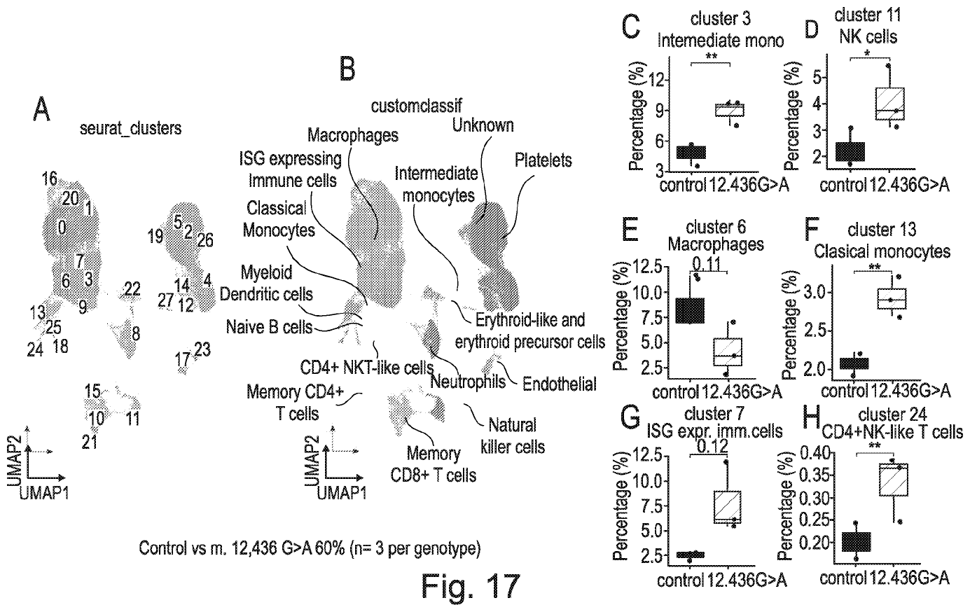

The inventors then profiled tumours using flow cytometry (see FIG. 16). Significantly altered populations are shown-NK cells, TAMs and Immature monocyte tumour residency appear to be differentially modulated by tumour mtDNA VAF. Single cell RNA sequencing further supported these data, demonstrating multiple macrophage, monocyte and NK cell resident populations that are differentially regulated by presence of high VAF mtDNA mutation (see FIG. 17).

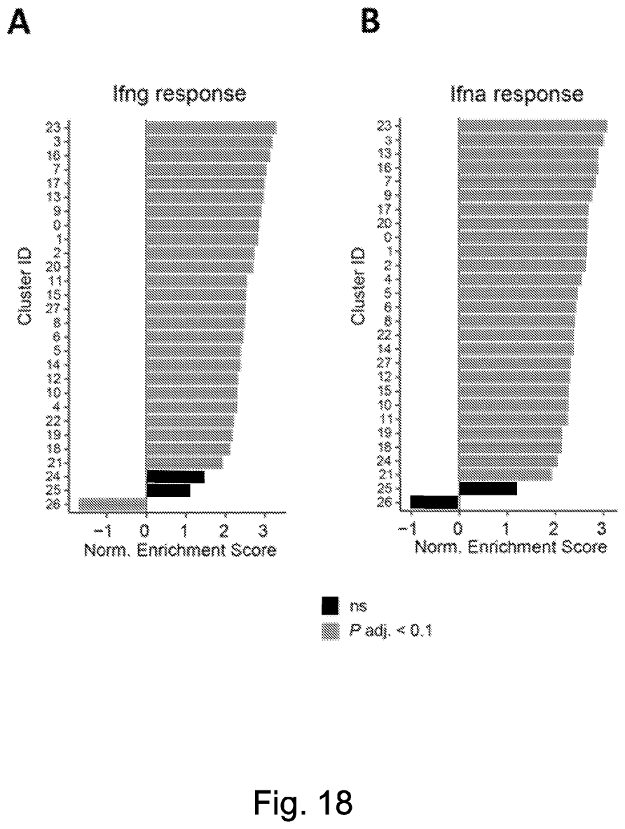

These changes in resident immune cells is coupled to a pan tumour interferon stimulated gene response (see FIG. 18), which is thought to be due to natural killer cells and CD4+NK-like T cells are the predominant source of interferon gamma. Interestingly, the only cell populations not demonstrating interferon gamma response are cluster 24 and 25, which are CD4+NK-like T cells and myeloid dendritic cells respectively. Dendritic cells are also the major source of interferon alpha, and interestingly cluster 25 is also one of only two populations not demonstrating significantly enhanced interferon alpha response.

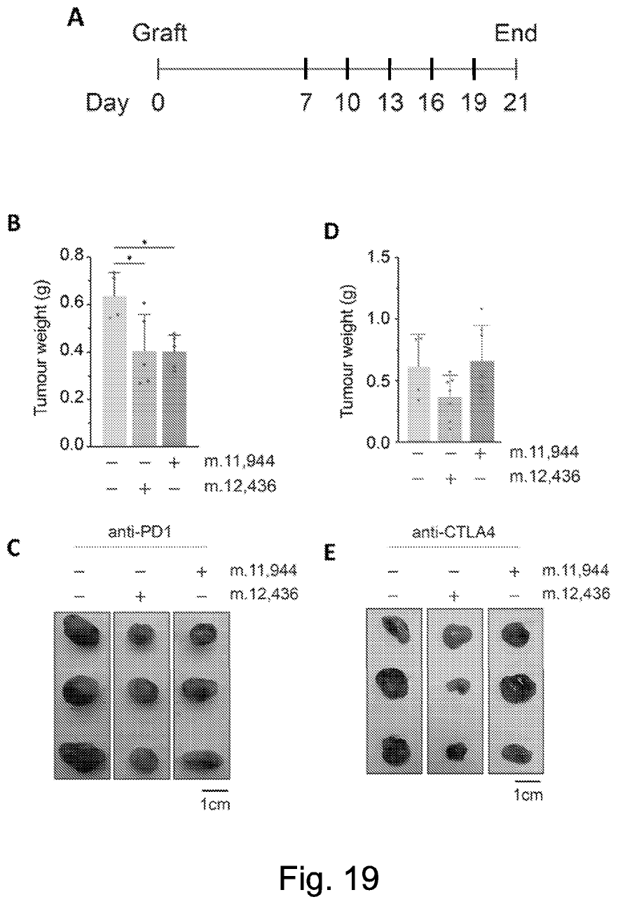

The inventors then sought to analyse the effect of this on how the mice would respond to replicate this in our mice using therapy (e.g. checkpoint blockade such as anti-PD1 treatment or anti-CTLA-4 treatment). Surprisingly, it was shown that high VAF mtDNA mutant melanoma tumours are differentially sensitive to anti-PD1 monoclonal antibody treatment in this extremely aggressive model of murine melanoma (FIG. 19). However, they are not sensitive to treatment with anti-CTLA4 monoclonal antibody treatment.

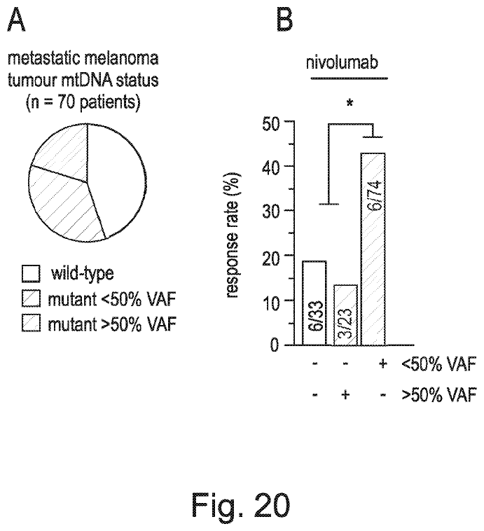

The inventors then assessed whether this would be associated with clinical outcome. Taking a small clinical cohort study (Riaz et al., 2017) they identified mtDNA mutant tumours with >50% VAF of pathogenic mtDNA mutations and calculated that these are 2.5× more likely to respond to nivolumab (anti-PD1) immunotherapy than mtDNA wild-type or low VAF tumours, with 40% of >50% VAF tumours responding compared with 17% of <50% VAF tumours responding (see FIG. 20). Of the total metastatic melanoma cohort treated with nivolumab (n=70) only 15 patients had a response to therapy. Of those 15, 12 were partial responses and 3 were complete responses. Surprisingly, 2 out of the 3 complete responders were categorised herein as >50% VAF.

The inventors have therefore identified a novel way of identifying cancer subjects that are likely to benefit from anti-PD1 therapy.

### Material & Methods

### 1. Maintenance of Cell Lines

B78 melanoma cells were cultured in standard Dulbecco's Modified Eagle Medium (DMEM) (Gibco), which contains 4.5 g/L glucose and 110 mg/L sodium pyruvate, with 20% fetal bovine serum (FBS) (Gibco), 1% Penicillin-Streptomycin (Gibco), 1× GLUTAMAX (Gibco) and 100 μg/mL uridine (Sigma). Cells were incubated at 37° C. and 5% CO₂ and split when ˜80% confluent.

### 2. Animal Models

All animal experiments were carried out in accordance with the UK Animals (Scientific Procedures) Act 1986 under PPL P72BA642F. The C57BL/6 mice were used for all studies and housed in up to 5 per cage in a temperature-controlled (21° C.) room with a 12-h light-dark cycle. Male mice of an average age of 6 weeks were used.

0.25×10⁶ cells were resuspended 1:1 in RPMI (Gibco) and Matrigel® Matrix (Corning) in 50 μL. Cells were injected over the flank and mice were culled at the tumour endpoint of 15 mm. Mice receiving checkpoint blockade therapy were dosed with 200 mg of Ultra-LEAF™ Purified anti-mouse CD279 (PD-1) (Biolegend) via intraperitoneal injections. Mice were dosed at 7 days post-injection of tumour cells and dosed twice a week till day 21 post-injection when they were culled, and tumours harvested.

### 3. Constructs and Plasmids

Transcription activation-like effector (TALE) domains were designed. TALE domains were cloned into either a pcmCherry or pTracer backbone.

4. Primers and siRNA

### PyroMark Assay Primers for Measuring Mt. 11944 Heteroplasmy

Forward:
(SEQ ID NO: 1)
CTTCATTATTAGCCTCTTAC
Reverse:
(SEQ ID NO: 2)
GTCTGAGTGTATATATCATG
Sequencing:
(SEQ ID NO: 3)
CTATTGAATTTATGGTGACT

### PyroMark Assay Primers for Measuring Mt. 11944 Heteroplasmy

Forward:
(SEQ ID NO: 4)
ATATTCTCCAACAACAACG
Reverse:
(SEQ ID NO: 5)
GTTATTATTAGTCGTGAGG
Sequencing:
(SEQ ID NO: 6)
CTATTGCTGATGGTAGT

ddPCR EvaGreen Primers

ND5 Forward:
(SEQ ID NO: 7)
TGCCTAGTAATCGGAAGCCTCGC
ND5 Reverse:
(SEQ ID NO: 8)
TCAGGCGTTGGTGTTGCAGG
VDAC1 Forward:
(SEQ ID NO: 9)
CTCCCACATACGCCGATCTT
VDAC1 Reverse:
(SEQ ID NO: 10)
GCCGTAGCCCTTGGTGAAG

siRNA for Metabolomics Experiments
ON-TARGETplus Mouse MDH1 siRNA-SMARTPool (L-0512-01-0005)
ON-TARGETplus Non-targeting Control Pool (D-001810-10-05)

### 5. Antibodies

### Primary and Secondary Antibodies for Immunoblotting and BN-PAGE

Total OXPHOS Rodent WB Antibody Cocktail (ab110413) used 1:800
MDH1 Polyclonal Antibody (15904-1-AP) used 1:1000
IRDye® 800CW Goat anti-Rabbit IgG (Licor) used 1:10,000
IRDye® 680RD Donkey anti-Mouse IgG (Licor) used 1:10,000

### Antibodies for Flow Cytometry

All antibodies were purchased from Biolegend and are anti-mouse.

TABLE 1
Neutrophil, Eosinophil, Monocyte and Macrophage Panel

| Target | Fluorophore |
|---|---|
| CD45 | A700 |
| CD11b | FITC |
| F4180 | BV510 |
| CD11c | BV785 |
| MHCII | BV421 |
| Ly6c | Perpcy5.5 |
| CD115 | BV711 |
| CX3CR1 | BV605 |
| SiglecF | A647 |
| Gp38 | PE |
| CD31 | Pecy7 |
| Epcam | BV650 |

TABLE 2
T-cell Panel

| Target | Fluorophore |
|---|---|
| CD3 | BV605 |
| CD45 | BV421 |
| Nk1.1 | Perpcy5.5 |
| PD-1 | BV510 |
| CD69 | BV650 |
| CD44 | BV711 |
| CD8 | FITC |
| CD4 | Pecy7 |
| B220 | BV785 |
| CD622 | A700 |
| CD27 | APC |
| CD3 | BV605 |

### 6. Cell Transfection and FACS

B78 cells were plated into 10 cm dishes to achieve ˜50% confluency on the day of transfection. 20 μg of DNA was mixed with 40 μL of P3000™ reagent and combined with 30 μL of Lipofectamine™ 3000 in a final volume of 1000 μL of OptiMEM. Transfection reagents were bought from Invitrogen. A negative control was set up alongside and the mixtures incubated at room temperature for 15-20 minutes before adding to the dishes. Cells were incubated at 37° C. and 5% CO2 for 24 hrs.

Cells were prepared for fluorescence-activated cell sorting (FACS) in 1 ml of DMEM and 1 μg/mL 4′,6-Diamidino-2-Phenylindole, Dihydrochloride (DAPI). Live cells were sorted for co-expression of mCherry and GFP and left to recover for 10 days before heteroplasmy measurements.

### 7. DNA Extraction

Cell culture medium was aspirated, and cells were washed once with PBS. Cells were detached using 1× trypsin (Gibco), re-suspended in cell culture medium and centrifuged at 300 g for 5 minutes. The pellet was re-suspended in 200 μL PBS for DNA extraction using the DNeasy Blood and Tissue Kit (Qiagen), according to the manufacturer's instructions. DNA concentration was then measured using a NanoDrop.

Tumour tissue (up to 20 mg) was treated as per the manufacturer's instructions using the DNeasy Blood and Tissue Kit (Qiagen).

### 8. Pyromark PCR

40 ng of genomic DNA extracted from cells, as per section 7, was mixed with 12.5 μL 5× PyroMark PCR Master Mix, 0.05 μL of 100 μM forward and reverse primers, 2.5 μL CoralLoad Concentrate and water to a final volume of 25 μL. All reagents were bought from Qiagen. PCR was performed according to the manufacturer's instructions with 60° C. to anneal.

The pyromark assay was designed using the PyroMark Assay Design 2.0 software. The assay was performed on the PyroMark Q48 Autoprep as per the manufacturer's instructions using 10 μL of each PCR product.

9. Digital Droplet PCR (ddPCR)

1 ng/μL sample DNA was mixed with 10 μL ddPCR Supermix for EvaGreen (2×) (BioRad), 110 nM of forward and reverse primers and water for a final volume of 20 μL per well. Samples were prepared in triplicate in a 96-well plate. The plate was sealed at 180° C. for 10 seconds using the PX1™ PCR Plate Sealer (Bio-Rad) and centrifuged briefly to remove any air bubbles. An Automated Droplet Generator (Bio-Rad) was used for droplet formation in a new 96-well plate. The plate was re-sealed and placed into a C1000 Touch Thermal Cycler (Bio-Rad) for PCR. PCR was performed according to the Bio-Rad ddPCR protocol for EvaGreen. Once completed, DNA was quantified using a QX200™ Droplet Reader (Bio-Rad).

### 10. Immunoblotting

Cultured cells were detached and spun down at 1000 g. The pellet was washed once with PBS and kept on ice. An appropriate amount of lysis buffer [10 mL radioimmunoprecipitation assay (RIPA) buffer (Invitrogen), 100 μL 1% Triton X-100 (Invitrogen) and 100 μL Halt™ Protease and Phosphatase Inhibitor Single-Use Cocktail (100×) (Invitrogen)] was added each pellet and left on ice for 10 minutes. The lysate solutions were spun down at 14,000 g for 5 minutes at 4° C. Protein quantification was done using the Pierce BCA Protein Assay Kit (Invitrogen) as per the manufacturer's instructions in a 96-well plate.

Protein samples were made to a final concentration of 100 μg in 50 μL. The appropriate amount of supernatant, based on the BCA assay, was mixed with 1:4 total volume of NuPAGE™ LDS Sample Buffer (4×) (Invitrogen) and 1:10 total volume of NuPAGE™ Sample Reducing Agent (10×) (Invitrogen). Samples were incubated at 37° C. for 20 minutes for and then loaded into Bolt™ 4-12% Bis-Tris Plus Gel (Invitrogen) with 1× MOPS buffer. PageRuler™ Prestained Protein Ladder (Invitrogen) was used. Gels were run at 180V until the dye front reached the end of the gel.

Once run, proteins were transferred onto a nitrocellulose membrane via wet transfer. The gel was placed in a ‘transfer sandwich’ in the order of: sponge, filter paper, gel, nitrocellulose membrane, filter paper and sponge. The transfer was run at 100V for 1 hour using 25 mM Tris, 192 mM glycine (pH 8.3) and 20% methanol in water as the buffer. The membrane was then washed in 1×TBST and then blocked with 5% non-fat milk in 1× TBST for 1 hour at room temperature on a roller. The solution was then replaced with the primary antibodies made in 5% non-fat milk in 1×TBST. The membrane was left overnight at 4° C. on a roller. The following day, the membrane was washed three times with 1×TBST for 5 minutes on a roller at room temperature before adding the secondary antibodies in 1×TBST. The membrane was covered and incubated on a roller for 1 hour at room temperature. The membrane was then washed three times with 1×TBST for 5 minutes before imaging on the Licor Odyssey Fc Imaging System.

### 11. Blue Native-PAGE

### 11.1 Mitochondrial Isolation

Cells were bulked to yield ˜100×10⁶ cells for mitochondrial isolation. Cells were trypsinised and pelleted into a 15 ml falcon tube. Cell pellets were washed twice in ice-cold PBS and spun at 600 g between each step before re-suspension in one-half volume of Hypotonic Buffer IB 0.1 (3.5 mM Tris-HCl PH 7.8, 2.5 mM NaCl, 0.5 mM MgCl₂). The cell suspension was homogenised with 80-100 strokes using a dounce homogeniser. 1/10th the original volume of packed cells of Hypertonic Buffer IB 10 (0.35M Tris-HCl PH 7.8, 0.25M NaCl, 50 mM MgCl₂) was immediately added to the suspension and the homogenate was transferred into a clean 15 ml falcon tube. Isotonic buffer IB 1 (35 mM Tris-HCl PH 7.8, 25 mM NaCl, 5 mM MgCl₂) was used to clean the homogeniser to collect excess cells and added to the homogenate. Samples were spun down at 12,000 g for 3 minutes at 4° C. to eliminate nuclear contamination. The supernatant was transferred to a clean tube and this step was repeated to ensure minimal contamination. The supernatant was then spun down 17,000 g for 2 minutes at 4° C. to pellet mitochondria. The mitochondrial pellet was then washed using Homogenisation Media (0.32M sucrose, 10 mM Tris-HCl PH 7.4, 1 mM EDTA) and spun again. The mitochondrial fractions were then used immediately for BN-PAGE.

### 11.2 BN-PAGE Gel and Imaging

All reagents were purchased through Invitrogen.

Mitochondrial pellets were solubilised in cold 1× NativePage™ Sample Buffer with 1% Digitonin. Samples were incubated on ice for 15 minutes and then spun down at 20,000 g for 30 minutes at 4° C. Protein concentration was determined using the Pierce™ BCA Assay Kit as per the manufacturer's instructions. Samples were made up to 100 μg in 50 ul with 1× NativePage™ Sample Buffer with 1% Digitonin. Immediately prior to loading the sample, NativePAGE 5% G-250 Sample Additive was added to each sample to a final concentration of 0.5%.

NativePage™ Novex 3-12% Bis-Tris Gels were used for this experiment. The cassette was removed and the wells were washed with Dark Blue Cathode Buffer (1× NativePage™ running buffer, 1× NativePage™ Cathode Additive in water). The gels were placed securely into an XCell SureLock Mini-Cell. The outer chamber was filled with ˜600 mL Anode Buffer (1× NativePage™ running buffer in 950 ml water) and ˜200 mL of the Dark Blue Cathode Buffer in the inner chamber. Samples were then loaded into the wells alongside the NativeMark™ Unstained Protein Standard. The gel was run at 150V and the Dark Blue Cathode Buffer was switched for the Light Blue Cathode Buffer (1× NativePage™ running buffer, 0.1× NativePage™ Cathode Additive in water) when the dye front had travelled ˜⅓^{rd} down. The gel was then left to run until the dye front had reached the bottom of the cell.

Proteins were transferred onto a PVDF membrane using the wet transfer method highlighted in section 11. The transfer buffer used in this experiment was 1× NuPage Transfer Buffer and the transfer was run at 60V for 1 hour.

The membrane was then blocked and blotted as per the method in section 10.

### 12. Seahorse Assay

The day prior to the assay, cells were plated at 20,000 cells/well in a Seahorse XF96 Cell Culture Microplate (Agilent). The outer rows and columns were left empty and cells were plated down each column. 200 μL of MQ water was also added to each well of the Seahorse XF96 sensor cartridge microplate and left alongside 50 mL of Seahorse XF Calibrant solution at 37° C. overnight.

The following day, the water from the Seahorse XF96 sensor cartridge microplate was discarded and replaced with 200 μL of the pre-heated calibrant. The cartridge was incubated at 37° C. for 45-60 minutes. Oligomycin, FCCP, rotenone and antimycin A were added to separate ports in the seahorse cartridge to a final concentration of 1 mM. The cartridge was then placed into the Seahorse XF96 Analyser for calibration.

In parallel, the cell culture plate was washed once with PBS. 150 μL of Seahorse media (25 mM glucose, 1 mM sodium pyruvate, 2 mM L-glutamine and 1% FBS in Seahorse XF Media) was added to each well and incubated at 37° C. for 30 minutes. The plate was then placed into the Seahorse XF96 Analyser following successful calibration. Oxygen consumption rate and extracellular acidification rate were measured using a Mito Stress template from the manufacturer's website.

### 13. Metabolomics

### 13.1 Experimental Media

Steady state metabolomics experiments used cell culture media, as per section 1, containing 2 mM L-glutamine (Gibco) in place of 1× GLUTAMAX. Plasmax was bought from Ximbio and supplemented with 2.5% dialysed FBS for these experiments.

U-¹³C glucose and 4-2H glucose were prepared using DMEM, no glucose (Gibco) and supplemented with 20% FBS, 1 mM sodium pyruvate (Gibco), 100 μg/mL uridine and either 25 mM U-¹³C glucose or 4-2H glucose.

U-¹³C glutamine and 1-¹³C glutamine medium was prepared using standard DMEM supplemented with 20% FBS, 100 μg/mL uridine and either 4 mM U-¹³C glutamine or 1-¹³C glutamine.

### 13.2 Intracellular and Media Metabolite Extraction

Cells were plated in triplicate in 12-well plates to achieve ˜70-80% confluency for the day of extraction. The following day, the media was aspirated and replaced with experimental media and the cells were incubated for 24 hours for extraction the following day.

All work was conducted on ice to reduce significant metabolite changes. 20 μL of media from each well was added to 980 μL ice-cold extraction media (50% LC/MS grade methanol, 30% LC/MS grade acetonitrile, 20% LC/MS grade water) for media analysis. The media from each plate was then quickly removed and washed twice with ice cold PBS. The plate was tapped on tissue paper and remaining PBS aspirated before adding 200 μL ice-cold extraction media to each well. The plate was stored at 4° C. and the extraction media was transferred into ice-cold microcentrifuge tubes. Samples were spun down at 14,000 g for 10 minutes at 4° C. before transferring into screw vials. Samples were stored at −80° C. until run on the mass spectrometer by the in-house facility.

Traces from all experiments were analysed using Tracefinder 4.0.

### 13.3 Normalisation of Samples

Plates used for the extractions were air-dried at room temperature and stored at 4° C. for a maximum of two weeks until the protein assay was performed.

The Lowry assay was used to measure protein concentration in each well. Briefly, 200 μL of Solution A (05% sodium deoxycholate and 1M sodium hydroxide in water) was added to each well, as well as an additional plate with a BSA standard curve, and left to shake vigorously at room temperature for 40 minutes. 2 mL of Solution B (0.629 mM copper disodium ethylenediaminetetraacetate, 189 mM sodium carbonate and 200 mM sodium hydroxide) and shake the plates for 10 minutes. 200 μL of Folin & Ciocalteu's phenol reagent (Sigma) was then added to each well and incubated at room temperature on a shaker for 40 minutes. 200 μL from each well was then transferred to a 96-well plate and the absorbance at 750 nm was read using a SpectraMax ABS Plus (Molecular Devices). The protein concentrations were calculated from the standard curve and used for normalisation of traces.

13.4 siRNA Knockdown

12,00 cells were plated per well of a 12-well plate for each siRNA experiment. Each condition was plated in triplicate. The following day, for each well, 5 μL of 5 μM siRNA was added to 95 μL Opti-MEM. In a separate tube, 5 μL DharmaFECT 1 Transfection Reagent (Horizon Discovery) was added to 95 μL Opti-MEM. The tubes were left at room temperature for 5 minutes to equilibrate before mixing. Samples were left at room temperature for 15-20 minutes. 800 μL of standard media was then added to the suspension and added to the cells. Experimental media was added 48 hours later and extractions were done as per section 14.2.

14. Cell and tissue bulk transcriptomics 1×10⁶ cells were pelleted into 1.5 ml microcentrifuge tubes and stored at −80° C. Tumour tissue (˜20 mg) was stored in RNAlater™ Stabilisation Solution (Invitrogen) and kept at −80° C. Samples were then sent on dry ice to Azenta for sample processing, sequencing and analysis.

### 15. Flow Cytometry

Harvested tumours (˜30 mg) were chopped and re-suspended in digestion buffer (500 U/mL collagenase I, 100 U/mL collagenase IV and 0.2 mg/mL DNase I in RPMI). Samples were incubated at 37 in a shaking rotor for 40 minutes. Samples were then passed through a 40 μm filter and spun down at 800 g for 3 minutes to pellet cells. Cells were re-suspended in 200 μL FACS buffer and split across two wells of a round-bottomed 96-well plate. The plate was spun down at the same speed and the supernatant was thrown off. Cell pellets were re-suspended in 100 μL 1:1000 Zombie Aqua (BioLegend) in PBS. The plate was kept at 4° C. for 20 minutes. The plate was re-spun and cell pellets were re-suspended in 100 μL of each flow panel made in FACS buffer, as outlined section 6. The plate was kept at 4° C. for at least 60 minutes. The plate was re-spun and the cell pellets were then re-suspended in 100 μL 4% Pierce™ 16% Formaldehyde (Invitrogen) and incubated at room temperature for 10 minutes. The plate was spun again, and samples were re-suspended in 100 μL of FACS buffer. The plate was wrapped in Parafilm and aluminium foil and kept at 4° C. for a maximum of 2 weeks.

Fixed samples were re-suspended in FACS buffer and moved to FACS tubes when needed to be run. 10×10⁶ events were recorded per sample on the Fortessa and analysis was done using FlowJo.

### 16. Tumour Single-Cell RNA Sequencing

Tumour tissue was digested as per section 15. Cells were then re-suspended in 1 ml of FACS buffer (2% FBS and 0.5 mM EDTA in PBS) with 1 μg/mL DAPI. Live cells were sorted and submitted to the in-house facility for barcoding using 10× Genomics Chromium platform and 3′ library prep kit. Sequencing was carried out at Glasgow Polyomics facility. Single-cell sequencing reads were aligned against the mouse GRCm39 reference genome using CellRanger (version 7.0.1), and the expression raw count matrixes were analyzed using the Seurate package (version 4.0.6). The malignant tumour cells and non-malignant cells were further separated based on the genome-wide copy number landscape changes estimated by the copykat package (version 1.1.0). The cell type of each cluster identified in the Seurate workflow was annotated using SingleR (version 1.10.0) through the gene expression correlation comparisons with known cell types in the mouse reference dataset. Differential gene expression analyses were carried out on log-normalized gene expression using the MAST algorithm within the FindMarkers function in Seurate.

### 17. Riaz et al Clinical Trial Data Analysis

Aligned reads for ChrM for patients' tumour and normal samples were acquired and processed according to previously described pipelines which are now publicly available (see e.g. Gorelick et al., Nature Metabolism 2021). Mutation calls and calculated heteroplasmy were used to assign patients to mtDNA wildtype, <50% VAF or >50% VAF groups.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### Sequences

### PyroMark Assay Primers for Measuring Mt. 11944 Heteroplasmy

Forward:
(SEQ ID NO: 1)
CTTCATTATTAGCCTCTTAC
Reverse:
(SEQ ID NO: 2)
GTCTGAGTGTATATATCATG
Sequencing:
(SEQ ID NO: 3)
CTATTGAATTTATGGTGACT

### PyroMark Assay Primers for Measuring Mt. 11944 Heteroplasmy

Forward:
(SEQ ID NO: 4)
ATATTCTCCAACAACAACG
Reverse:
(SEQ ID NO: 5)
GTTATTATTAGTCGTGAGG
Sequencing:
(SEQ ID NO: 6)
CTATTGCTGATGGTAGT

ddPCR EvaGreen Primers

ND5 Forward:
(SEQ ID NO: 7)
TGCCTAGTAATCGGAAGCCTCGC
ND5 Reverse:
(SEQ ID NO: 8)
TCAGGCGTTGGTGTTGCAGG
VDAC1 Forward:
(SEQ ID NO: 9)
CTCCCACATACGCCGATCTT
VDAC1 Reverse:
(SEQ ID NO: 10)
GCCGTAGCCCTTGGTGAAG

### REFERENCES

Gorelick et al., 2021, Nature Metabolism April; 3 (4): 558-570.
Riaz et al., 2017 doi: 10.1016/j.cell.2017.09.028. Epub 2017 Oct. 12.

### Example 2—mtDNA Mutations in Complex 4

Mt-Co1 is a mitochondrially encoded subunit of complex IV. The inventors made DddA-derived cytosine base editors (DdCBEs) to introduce a G>A point mutation in this protein at position m.6214 of the mouse mitochondrial genome.

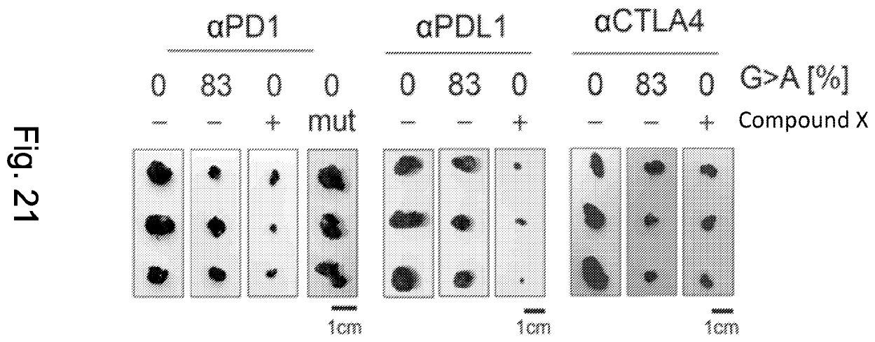

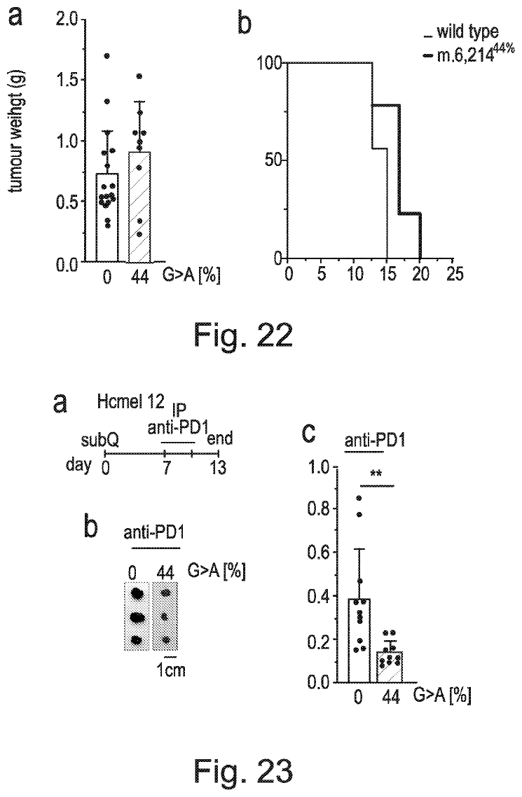

When implanted into BI6 mice these tumours grew at comparable rates to wild-type, reaching comparable endpoint weight in similar time (FIG. 22A-B).

When challenged with anti-PD1, the Mt-Co1 mutant tumours showed a clear reduction in size at endpoint relative to wildtype tumours. This heteroplasmy is notably lower than that required for a robust immune response for Mt-Nd5 truncation—and this is likely due to the more profound effect on the respiratory chain that loss of complex IV will cause (FIG. 23A-C).

These results show that mutations in complexes in other than complex I result in tumour sensitization to checkpoint inhibitors, further supporting that metabolic changes within the cells play an important role in increasing tumour sensitivity to checkpoint inhibitors.

### Example 3—Increasing Sensitivity to Anti-CTLA4 Treatment and Anti-PD-L1 Treatment

As seen in FIG. 21, 12,436⁸³% tumors were significantly smaller upon treatment with different types of immune checkpoint inhibitors (such as PD1 inhibitors, PD-L1 inhibitors, and CTLA4 inhibitors).

1. A method of predicting whether a subject having a cancer or a pre-cancer would benefit from treatment with an immune checkpoint inhibitor, comprising:
a) determining the deleterious mitochondrial DNA (mtDNA) mutation load in a cancer or pre-cancer sample from the subject; and
b) predicting that the subject would benefit from the treatment when the deleterious mtDNA mutation load is 30% or more.

2. An immune checkpoint inhibitor for use in treating a cancer or a pre-cancer in a subject, wherein the cancer or pre-cancer has a deleterious mtDNA mutation load of 30% or more.

3. A method of treating a cancer or a pre-cancer in a subject, comprising:
(i) determining the deleterious mtDNA mutation load in a cancer or pre-cancer sample from the subject; and
(ii) administering an immune checkpoint inhibitor if the subject has a deleterious mtDNA mutation load of 30% or more.

4. The method according to claim 1 or 3, or the inhibitor for use according to claim 2, wherein the deleterious mtDNA mutation load is 40% or more, 50% or more, or 60% or more.

5. The method, or inhibitor for use, according to any preceding claim, wherein the deleterious mtDNA mutation load increases the lactate to glucose ratio in the cancer or pre-cancer to above 3:1.

6. The method, or inhibitor for use, according to any preceding claim, wherein the deleterious mtDNA mutation load alters the immune micro-environment in the cancer or pre-cancer, by:
(i) increasing the level of NK cells;
(ii) decreasing the level of macrophages, optionally wherein the macrophages are in tumour associated macrophages; and/or
(iii) increasing the level of monocytes, optionally wherein the monocytes are immature, intermediate or classical monocytes;
(iv) increasing the level of CD4 NK-like T cells; and/or
(v) increasing the level of ISG-expressing immune cells.

7. The method, or inhibitor for use, according to any preceding claim, wherein the deleterious mtDNA mutation is selected from the group consisting of:
(i) a tRNA mutation having a MitoTIP RAW score of at least 12.6, or at least 16.25;
(ii) a rRNA mutation;
(iii) a truncation mutation in a mtDNA gene;
(iv) a missense mutation in a mtDNA gene, wherein the missense mutation has an Apogee score of more than 0.5, optionally wherein the missense mutation is selected from a frameshift mutation, an insertion mutation or a deletion mutation; and/or
(v) a mutation in a mtDNA D-loop region selected from the group consisting of: the H-strand promoter (545-567), MT-HV2 (hypervariable segment 2) m.57-372, and MT-HV1 (hypervariable segment 1)-m. 16024-16390.

8. The method, or inhibitor for use, according to any preceding claim, wherein the deleterious mtDNA mutation is in a gene selected from the group consisting of: MT-ND5, MT-ND1, MT-ND2, MT-ND3, MT-ND4, MT-ND4L, MT-ND6, MT-CO1, MT-CO2, MT-CO3, MT-CYB, MT-ATP6, MT-ATP8, MT-TL1, MT-TA, MT-TC, MT-TD, MT-TE, MT-TF, MT-TG, MT-TH, MT-TI, MT-TK, MT-TL2, MT-TM, MT-TN, MT-TP, MT-TQ, MT-TR, MT-TS1, MT-TS2, MT-TT, MT-TV, MT-TW, MT-TY, MT-RNR1 and MT-RNR2.

9. The method, or inhibitor for use, according to claim 8, wherein the ND5 deleterious mtDNA mutation is a truncating mutation that is in a region selected from: m. 12418-12425: A indel or m. 12385-12390: C indel.

10. The method, or inhibitor for use, according to any preceding claim, wherein the deleterious mtDNA mutation is a truncation, missense, insertion, or frameshift mutation.

11. The method, or inhibitor for use, according to any preceding claim, wherein the cancer or pre-cancer is selected from the group consisting of: skin, breast, colon, colorectal, oesophageal, thyroid, renal, stomach, ovarian, pancreatic and lung cancer or pre-cancer.

12. The method, or inhibitor for use, according claim 11, wherein the cancer or pre-cancer is selected from the group consisting of:
(i) a renal cancer or precancer, optionally wherein the renal cancer is a renal cancer papillary and chromophobe subtype;
(ii) a thyroid cancer or pre-cancer, optionally wherein the thyroid cancer is Hürthle cell carcinoma (HCC);
(iii) ovarian cancer or pre-cancer, optionally wherein the ovarian cancer is serous high grade ovarian (SHGO) cancer; and
(iv) colorectal cancer or pre-cancer, optionally wherein the colorectal cancer is colorectal adenocarcinoma.

13. The method, or inhibitor for use, according to claim 11, wherein the skin cancer is melanoma.

14. The method, or inhibitor for use, according to any preceding claim, wherein the immune checkpoint inhibitor is selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor, optionally wherein the immune checkpoint inhibitor is selected from the group consisting of a PD-1 inhibitor, a PD-L1 inhibitor, and CTLA4 inhibitor, further optionally wherein the PD-1 inhibitor is nivolumab.

15. A method of predicting whether a subject having a cancer or a pre-cancer would benefit from treatment with an immune checkpoint inhibitor, comprising:
a) determining the redox status in a cancer or pre-cancer sample from the subject; and
b) predicting that the subject would benefit from the treatment when there is an altered redox status.

16. An immune checkpoint inhibitor for use in treating a cancer or a pre-cancer in a subject, wherein the cancer or pre-cancer has an altered redox status.

17. A method of treating a cancer or a pre-cancer in a subject, comprising:
(i) determining the redox status in a cancer or pre-cancer sample from the subject; and
(ii) administering an immune checkpoint inhibitor if the subject has an altered redox status.

18. The method of claim 15, the immune checkpoint inhibitor for use according to claim 16, the method of claim 17, wherein the redox status is determined by determining the lactate to glucose ratio.

19. The method, or inhibitor for use according to claim 18, wherein the subject has an elevated lactate to glucose ratio in the cancer or pre-cancer.

20. The method, or inhibitor for use, according to claim 19, wherein the elevated lactate to glucose ratio in the cancer or pre-cancer is above 3:1.

21. The method, or inhibitor for use, according to any one of claims 19 to 20, wherein the elevated lactate to glucose ratio alters the immune micro-environment in the cancer or pre-cancer, by:
(i) increasing the level of NK cells;
(ii) decreasing the level of macrophages, optionally wherein the macrophages are in tumour associated macrophages; and/or
(iii) increasing the level of monocytes, optionally wherein the monocytes are immature, intermediate or classical monocytes;
(iv) increasing the level of CD4 NK-like T cells; and/or
(v) increasing the level of ISG-expressing immune cells.

22. The method, or inhibitor for use, according any one of claims 15 to 21, wherein the cancer or pre-cancer is selected from the group consisting of: skin, breast, colon, colorectal, oesophagus, thyroid, renal, stomach, ovaries, pancreas and lung cancer or pre-cancer.

23. The method, or inhibitor for use, according claim 22, wherein the cancer or pre-cancer is selected from the group consisting of:
(i) a renal cancer or precancer, optionally wherein the renal cancer is a renal cancer papillary and chromophobe subtype;
(ii) a thyroid cancer or pre-cancer, optionally wherein the thyroid cancer is Hürthle cell carcinoma (HCC);
(iii) ovarian cancer or pre-cancer, optionally wherein the ovarian cancer is serous high grade ovarian (SHGO) cancer; and
(iv) colorectal cancer or pre-cancer, optionally wherein the colorectal cancer is colorectal adenocarcinoma.

24. The method, or inhibitor for use, according to claim 22, wherein the skin cancer is melanoma.

25. The method, or inhibitor for use, according to any one of claims 15 to 24, wherein the immune checkpoint inhibitor is selected from the group consisting of a PD-1 inhibitor, a PD-L1 inhibitor, a PD-L2 inhibitor, CTLA4 inhibitor, TIGIT inhibitor, LAG-3 inhibitor, TIM-3 inhibitor, BTLA inhibitor and KIR inhibitor, optionally wherein the immune checkpoint inhibitor is selected from the group consisting of a PD-1 inhibitor, a PD-L1 inhibitor, and CTLA4 inhibitor, further optionally wherein the PD-1 inhibitor is nivolumab.


SEQUENCE LISTING (12 sequences)

SEQ ID NO: 1 - DNA, 20 bp, synthetic construct
  source 1..20: mol_type = other DNA
  misc_feature 1..20: note = PyroMark assay primers for measuring mt.11944 heteroplasmy - forward
cttcattattagcctcttac

SEQ ID NO: 2 - DNA, 20 bp, synthetic construct
  source 1..20: mol_type = other DNA
  misc_feature 1..20: note = PyroMark assay primers for measuring mt.11944 heteroplasmy - reverse
gtctgagtgtatatatcatg

SEQ ID NO: 3 - DNA, 20 bp, synthetic construct
  source 1..20: mol_type = other DNA
  misc_feature 1..20: note = PyroMark assay primers for measuring mt.11944 heteroplasmy - sequencing
ctattgaatttatggtgact

SEQ ID NO: 4 - DNA, 19 bp, synthetic construct
  source 1..19: mol_type = other DNA
  misc_feature 1..19: note = PyroMark assay primers for measuring mt.11944 heteroplasmy - forward
atattctccaacaacaacg

SEQ ID NO: 5 - DNA, 19 bp, synthetic construct
  source 1..19: mol_type = other DNA
  misc_feature 1..19: note = PyroMark assay primers for measuring mt.11944 heteroplasmy - reverse
gttattattagtcgtgagg

SEQ ID NO: 6 - DNA, 17 bp, synthetic construct
  source 1..17: mol_type = other DNA
  misc_feature 1..17: note = PyroMark assay primers for measuring mt.11944 heteroplasmy - sequencing
ctattgctgatggtagt

SEQ ID NO: 7 - DNA, 23 bp, synthetic construct
  source 1..23: mol_type = other DNA
  misc_feature 1..23: note = ddPCR EvaGreen primers ND5 - forward
tgcctagtaatcggaagcctcgc

SEQ ID NO: 8 - DNA, 20 bp, synthetic construct
  source 1..20: mol_type = other DNA
  misc_feature 1..20: note = ddPCR EvaGreen primers ND5 - reverse
tcaggcgttggtgttgcagg

SEQ ID NO: 9 - DNA, 20 bp, synthetic construct
  source 1..20: mol_type = other DNA
  misc_feature 1..20: note = ddPCR EvaGreen primers VDAC1 - forward
ctcccacatacgccgatctt

SEQ ID NO: 10 - DNA, 19 bp, synthetic construct
  source 1..19: mol_type = other DNA
  misc_feature 1..19: note = ddPCR EvaGreen primers VDAC1 - reverse
gccgtagcccttggtgaag

SEQ ID NO: 11 - DNA, 45 bp, synthetic construct
  source 1..45: mol_type = other DNA
  misc_feature 1..45: note = Mt-Nd5 m. 12,436
tttggcctccacccatgactaccatcagcaatagaaggccctaca

SEQ ID NO: 12 - DNA, 45 bp, synthetic construct
  source 1..45: mol_type = other DNA
  misc_feature 1..45: note = Mt-Nd5 m. 11,944
taattacaacctggcactgagtcaccataaattcaatagaaaact